(19) United States
(12) Patent Application Publication
(10) Pub. No.: US 2020/0232011 A1
(43) Pub. Date: Jul. 23, 2020

(54) METHODS OF NUCLEIC ACID DETECTION AND PRIMER DESIGN

(71) Applicant: Mission Bio, South San Francisco, CA (US)

(72) Inventors: David Ruff, San Francisco, CA (US); Dalia Dhingra, San Francisco, CA (US)

(73) Assignee: Mission Bio, South San Francisco, CA (US)

(21) Appl. No.: 16/749,731

(22) Filed: Jan. 22, 2020

Related U.S. Application Data
(60) Provisional application No. 62/795,171, filed on Jan. 22, 2019.

(51) Int. Cl. C12Q 1/6806; C12Q 1/6853; C12Q 1/686

(52) U.S. Cl. CPC C12Q 1/6806; C12Q 2600/16; C12Q 1/686; C12Q 1/6853

(57) ABSTRACT

Provided herein are methods for detection of a target nucleic acid from a single cell. Preferred embodiments of the method include selecting one or more target nucleic acid sequence of interest in an individual cell, where the target nucleic acid sequence is typically complementary to cellular DNA, including a genomic DNA, and an RNA in a cell. A cell sample is provided, and in preferred embodiments the sample is from a single cell. The cell is lysed and in a single reaction both DNA and RNA can be detected without sub-dividing the sample. This can be accomplished by providing nucleic acid amplification primer sets complementary to one or more target nucleic acid, and in particular primer sets that selectively amplify particular target nucleic acids or amplicons in an amplification reaction. Also provided are methods of primer design for these methods and apparatus and system used to perform the methods.

### RELATED APPLICATIONS

This application takes priority to a U.S. Provisional Application U.S. Ser. No. 62/795,171 filed Jan. 22, 2019, by D. Dhingra and D. Ruff, entitled “Method, Systems and Apparatus for DNA and RNA Primer Design”.

### FIELD

This invention relates generally to the detection of target genes or nucleic acids in a cell or organism, and more particularly to the detection and identification of both DNA and RNA from one or more target nucleic acid in a single cell.

### SEQUENCE LISTING

The instant application contains a Sequence Listing which has been submitted electronically in ASCII format and is hereby incorporated by reference in its entirety. Said ASCII copy, created on Mar. 17, 2020, is named MBI-0300US_SL.bct and is 25,646 bytes in size.

### BACKGROUND

Nucleic acid analysis methods based on the complementarity of nucleic acid nucleotide sequences can analyze genetic traits directly. Thus, these methods are a very powerful means for identification of genetic diseases, identification and monitoring of cancer, microorganisms etc.

The detection of a target gene or nucleic acid present in a very small amount in a sample, such as from a single cell, is difficult and becomes even more problematic when multiple target nucleic acids comprising cellular DNA, including genomic, extrachromosomal, viral and mitochondrial DNA and RNA need to be analyzed.

There is a need for method, system and apparatus to provide high-throughput, single-cell nucleic acid sequencing that incorporates targeted RNA combined with targeted DNA sequencing. The inventions described herein meet these unsolved challenges and needs.

### BRIEF SUMMARY

The inventions described and claimed herein have many attributes and embodiments including, but not limited to, those set forth or described or referenced in this Brief Summary. The inventions described and claimed herein are not limited to, or by, the features or embodiments identified in this Summary, which is included for purposes of illustration only and not restriction.

In one aspect, the disclosed embodiments generally incorporate targeted RNA combined with targeted DNA sequencing. Certain embodiments provide substantially combined targeted-RNA and -DNA sequencing to single cell sequencing workflow. In one embodiment, the method requires substantially no sample splitting into RNA and DNA fractions. The amplification product (amplicon) may have overlapping coverage between the genome and transcriptome. Some embodiments provide methods of selective amplification of DNA or RNA amplicons, in part, by selecting primers with particular sequences or modifications of the primers. The DNA and RNA amplicons may also be distinguished through sequencing and balanced for optimal sequencing depth of each.

In another aspect, methods of designing and providing primers useful for the selective or preferential amplification of a DNA or RNA amplicon are provided. Amplification primers may also incorporate chemical modifications in the backbone, nucleotides, or otherwise that effect, for example reduce, prevent, or limit, the amplification of particular amplicons based on sequence or target nucleic acid type (e.g. mRNA or gDNA).

For example in some embodiments, primers are designed and provided where the DNA reverse primer is blocked so as not to be extended until PCR. In other embodiments, the DNA reverse primer and the forward primers are blocked. In other embodiments. an amplification reaction has the DNA reverse primer and the forward primers blocked so as not to be extended until PCR.

Certain embodiments utilize solid beads having an alternate chemistry where the forward primers to be used for both DNA and RNA are in solution. In these embodiments, forward primers contain a PCR annealing sequence embedded, or ‘handle’, that allows hybridization to primers. The handle is a specific tail 5′ upstream of the target sequence. This handle is complimentary to bead barcoded oligo and serves as a PCR extension bridge to link the target amplicon to the bead barcode library primer sequence. The solid beads contain primers that can anneal to the PCR handle on the forward primers. The gene specific RNA reverse primers and gene specific DNA reverse primers are in solution. The RNA reverse primer can be used for reverse transcription. In particular embodiments, the DNA reverse primer is blocked so as not to be extended until PCR. The methods described herein are effectively unlimited with respect to the number of unique nucleic acids labels that can be generated.

The workflow of an exemplary embodiment involves loading cells on an instrument to release the genomic DNA and RNA (nucleic acids). The released nucleic acids are then introduced to reagents configured for reverse transcription and PCR. In one embodiment, solid beads may be used for this purpose. Here, the beads are loaded with forward primers to be used for both DNA and RNA with all reverse primers in solution—gene specific RNA reverse primers and gene specific DNA reverse primers. The RNA reverse primer can be used for reverse transcription. The high throughput nature of the methods described herein allow multiomic analysis of DNA and RNA to be performed on thousands to millions of single cells, providing a scalable means by which to characterize the nucleic acids of large numbers of single cells.

In another aspect, methods for detection of a target nucleic acid from a single cell are provided. A non-limiting representative embodiment includes, independent of order presented, many or all of the following steps: selecting one or more target nucleic acid sequence of interest in an individual cell, wherein the target nucleic acid sequence is complementary to a nucleic acid in a cell; providing a sample having a plurality of individual single cells; encapsulating one or more individual cell(s) in a reaction mixture comprising a protease; incubating the encapsulated cell with the protease in the drop to produce a cell lysate; providing one or more nucleic acid amplification primer sets, where each primer set is complementary to a target nucleic acid and at least one primer of a nucleic acid amplification primer set includes a barcode identification sequence; performing a nucleic acid amplification reaction to form an amplification product from the nucleic acid of a single cell, where the amplification product includes amplicons of one or more target nucleic acid sequence; providing an affinity reagent that includes a nucleic acid sequence complementary to the identification barcode sequence of one of more nucleic acid primer of a primer set, wherein said affinity reagent comprising said nucleic acid sequence complementary to the identification barcode sequence is capable of binding to a nucleic acid amplification primer set comprising a barcode identification sequence; contacting an affinity reagent to the amplification product comprising amplicons of one or more target nucleic acid sequence under conditions sufficient for binding of the affinity reagent to the target nucleic acid to form an affinity reagent bound target nucleic acid; and determining the identity of the target nucleic acids by sequencing the first bar code and second bar code.

A target nucleic acid is typically either DNA or RNA. In some embodiments, amplification products are produced from both DNA and RNA target nucleic acid sequences.

Certain embodiments include the addition of a reverse transcriptase polymerase and a step of producing cDNA from an RNA target sequence where an mRNA target nucleic acid from a single cell is detected and identified.

In another embodiment, each primer set provided includes a forward primer and a reverse primer that are complementary to a target nucleic acid or the complement thereof.

In another embodiment a forward primer of a primer set includes an identification barcode sequence.

In one embodiment, one or more nucleic acid amplification primer sets provided comprise a DNA specific primer that is blocked before reverse transcriptase is added. One implementation of this embodiment includes providing a DNA reverse primer that is blocked during any reverse transcriptase activity so that cDNA is only created by a RNA reverse primer. In another implementation, a DNA reverse primer that is outside of the RNA reverse primer is provided so that cDNA is only extended by a RNA reverse primer.

In one embodiment, the target nucleic acid may comprise both DNA and RNA, and either DNA or RNA is selectively amplified to form an amplicon product specific for either a DNA or an RNA target nucleic acid.

In one embodiment, DNA or RNA amplicons are attenuated, limited, or prevented during amplification by using competimers that selectively amplify DNA or RNA amplicons.

In another embodiment, DNA or RNA amplicons are attenuated, limited, or prevented during amplification by using biotinylated primers that selectively amplify DNA or RNA amplicons.

In another embodiment, a portion of amplification primers provided for RNA amplification comprise uracil and enable the removal RNA amplicons by cleavage.

In another embodiment, a method for detection of a target nucleic acid from a single cell includes, independent of order presented, the following: selecting one or more target nucleic acid sequence of interest in an individual cell, where the target nucleic acid sequence is complementary to a genomic DNA and an RNA in a cell; providing a sample having a plurality of individual single cells; encapsulating one or more individual cell(s) in a reaction mixture comprising a protease; incubating the encapsulated cell with the protease in the drop to produce a cell lysate; providing one or more nucleic acid amplification primer sets complementary to one or more target nucleic acid, wherein at least one primer of a nucleic acid amplification primer set includes a barcode identification sequence and wherein one or more nucleic acid amplification primer sets provided comprise a DNA specific primer; adding a reverse transcriptase polymerase and producing cDNA from an RNA target; performing a nucleic acid amplification reaction to form an amplification product from the nucleic acid of a single cell, said amplification product comprising amplicons of one or more target nucleic acid sequence.

Implementations of the embodiment above may further include i) providing an affinity reagent that includes a nucleic acid sequence complementary to the identification barcode sequence of one of more nucleic acid primer of a primer set, wherein said affinity reagent comprising said nucleic acid sequence complementary to the identification barcode sequence is capable of binding to a nucleic acid amplification primer set comprising a barcode identification sequence, and ii) contacting an affinity reagent to the amplification product having amplicons of one or more target nucleic acid sequence under conditions sufficient for binding of the affinity reagent to the target nucleic acid to form an affinity reagent bound target nucleic acid and determining the identity of the target nucleic acids by sequencing the first bar code and second bar code.

In another aspect, methods of designing primers for the amplification of target nucleic acids by methods described herein are provided. An exemplary method of primer design for selective detection of nucleic acids in a sample having both genomic DNA and mRNA includes, irrespective of order, the following steps: selecting a target nucleic acid sequence of interest in an individual cell, where the target nucleic acid sequence is complementary to a mRNA of potential interest that has a corresponding genomic DNA of potential interest; selecting and providing a DNA reverse primer that is blocked to be incapable of priming and extension by reverse transcriptase; selecting and providing one or more nucleic acid amplification primer sets complementary to one or more target nucleic acid, where at least one primer of a nucleic acid amplification primer set includes a barcode identification sequence and where one or more nucleic acid amplification primer sets provided include a DNA specific primer; and, optionally, selecting and providing a DNA reverse primer that is outside of the RNA reverse primer in a target nucleic acid region to be amplified; and, optionally, selecting and providing competing competimer primers that selectively amplify DNA or RNA amplicons.

### BRIEF DESCRIPTION OF THE DRAWINGS

FIG. 1 schematically illustrates an exemplary RNA plus DNA amplification embodiment. Amplicons have same tails for library PCR. They can be distinguished from their start sites from Read 2. RNA amplicons can be attenuated during library PCR using competimers or biotinylated primers that selectively amplify DNA or RNA amplicons. A percent of library primers for RNA could also be synthesized with uracil so we can remove RNA library molecules with cleavage.

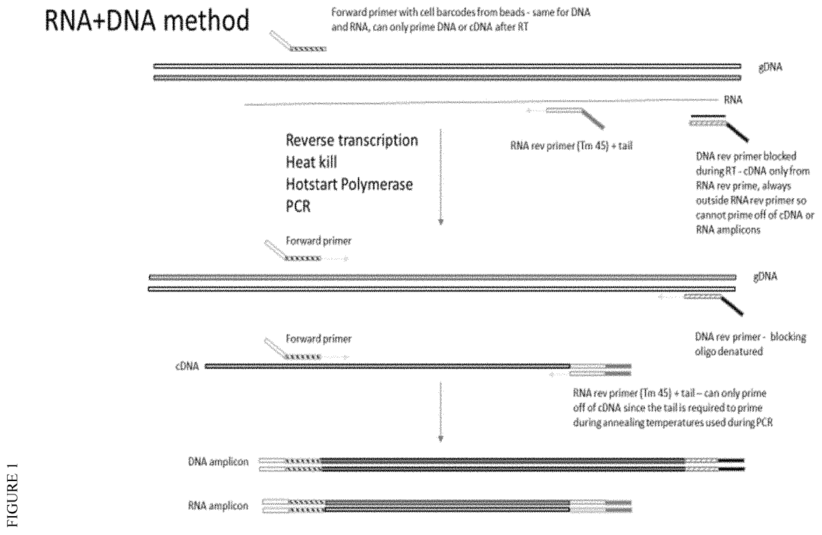

FIG. 2 schematically illustrates an exemplary ddNTP amplification embodiment. Amplicons have same tails for library PCR. They can be distinguished from their start sites from Read 2. RNA amplicons can be attenuated during library PCR using competimers or biotinylated primers that selectively amplify DNA or RNA amplicons. A percent of library primers for RNA could also be synthesized with uracil so we can remove RNA library molecules with cleavage.

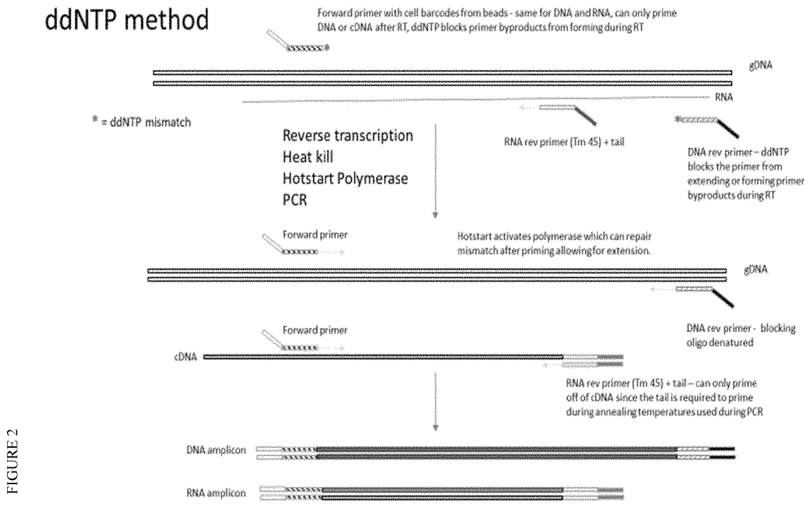

FIG. 3 schematically illustrates samples primer interactions. Primer interactions from the new DNA primers will occur if multiplexed. In this diagram (left) the THSP_HRAS_1_fwd with THSP_APC_1_fwd primer 5′-(CAAATGAAAACCAAGAGAAAGAGGC SEQ ID NO: 1)) is shown hybridizing with THSP_HRAS_1_fwd primer (GGATGTCCTCAAAGACTTGGTGT SEQ ID NO: 2)). The THSP_HRAS_1_fwd with THSP_PTEN_2_fwd primer 5′-(GTAAATACATTCTTCATACCAGGACCAGAG (SEQ ID NO: 3)) is shown hybridizing with 5′-(GGATGTCCTCAAAAGACTTGGTGT (SEQ ID NO: 4). Similar interactions were observed with RNA primers alone. Also show (right) are show samples of RNA primer interactions. The primer 5′ (GTAAATACATTCTTCATACCAGGACCAGAG (SEQ ID NO: 3) hybridizing with 5′-(TTTGCAGGGTATTA (SEQ ID NO: 5) and 5′-(CCTGTTGGACATC (SEQ ID NO: 6) with 5′ (CACCATGATGTGC SEQ ID NO: 7)). FIG. 3 also discloses SEQ ID NOS 12, 10, and 28, respectively, in order of appearance.

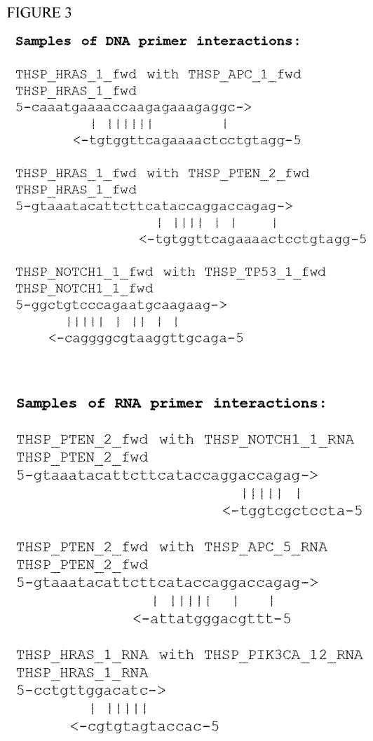

FIG. 4 shows an exemplary forward primer design where forward primers are the same as V1 chemistry with the primers on the beads. The same forward primers are used for DNA and RNA. Bulk reactions will be performed with the same tail as the forward primers on the bead.

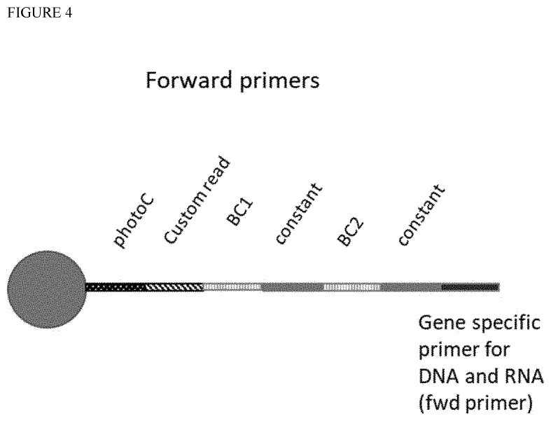

FIG. 5 illustrates an SNP check. Only NOTCH1_1 (SEQ ID NOS 10 and 103-105, respectively, in order of appearance) and PIK3CA_12 (SEQ ID NOS 11 and 106-108, respectively, in order of appearance) had SNPs under the RNA reverse primers. They were redesigned to move the site further from the 3′ end. The primers were designed using specific Tm requirements. The reverse transcriptase primers were designed to have a Tm in the range of 42-48° C. (lower primer in FIG. 5). The opposite PCR primers, forward primers, were designed to have higher Tms in the range of 58-64° C. The first reaction in this process is catalyzed by reverse transcriptase and the reaction is conducted at an optimal temperature between 37-50° C. The RNA molecule can only be primed by the lower primer to generate the first-strand of cDNA. The upper, forward, primer is used to generate the second-strand and then both primers participate in PCR amplification. An integral requirement for primer design is to ensure no common SNPs are present in the target sequence that hybridizes to the primers. Primers can be screened against common human genome databases such as the UCSC genome browser to fulfill this process. FIG. 5 displays an exemplary design that has the primers surround a target region that possesses the SNPs to be interrogated.

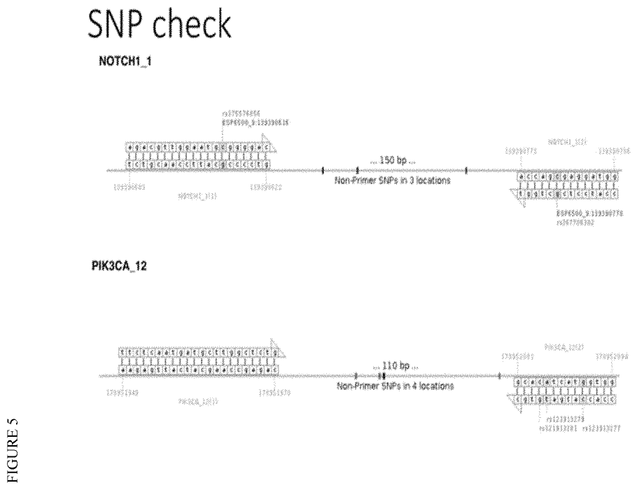

FIG. 6 shows the results from an RNA amplification, RT-qPCR. The amplification reaction mixture included the following: 5 μL 2× MasterMix; 0.2 μL 10 μM RNA rev; 0.4 μL 10 μM fwd; 0.25 μL Superscript RT; 1.5 μL RNA; 0.5 μL, Evagreen; 0.2 μL ROX; and 0.43 μL water. In this graph the Y axis shows the amount of amplification product as measured by fluorescence and the X-axis shows the number of amplification cycles. In this embodiment 15 ng of RNA was used as an input. The primers utilized were THSP_PTEN_2_RNA_rev_seq+THSP_PTEN_2_fwd_seq in SuperScript IV One-Step RT-PCR System. As each qPCR cycle amplifies target, the SYBR Green dye fluorescence is measured. The qPCR cycling parameters are displayed in the table. Once sufficient PCR amplification cycles generate an amount of amplicon product above the detection threshold, the qPCR instrument (Agilent) displays fluorescent amplification curve. When this amplification curves crosses a threshold line (Y-axis), that cycle number (X-axis) is called the threshold cycle (C_{T}).

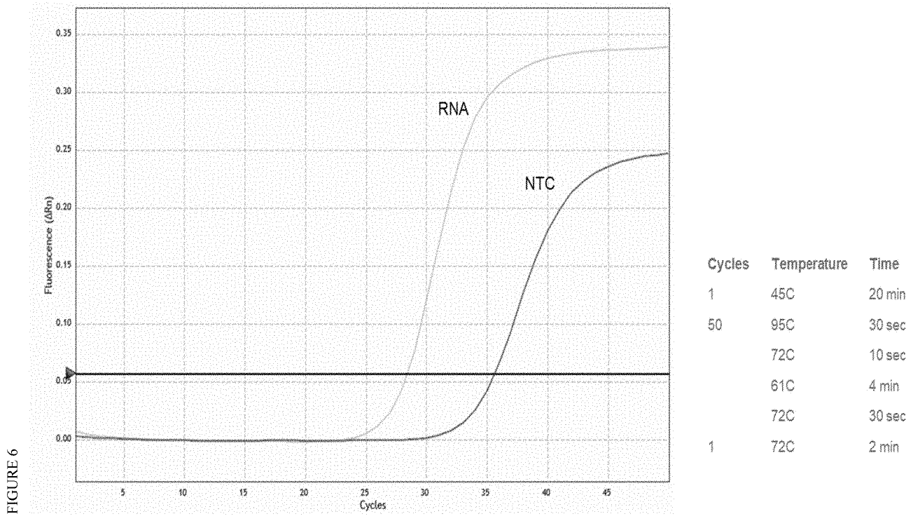

FIG. 7 shows products from the RNA amplification shown in FIG. 6. The Y axis shows the amount of amplification product in each peak as measured in fluorescence units, while the X axis shows the size or length of the amplicons in nucleotide base pairs. The qPCR product from amplification is analyzed on a Bioanalyzer DNA 1000 chip; 1:10 dilution; Expected THSP_PTEN_2RNA amplicon=149 bp. This Bioanalyzer displays a single PCR product from the sample of approximately 149-154 base pairs in size.

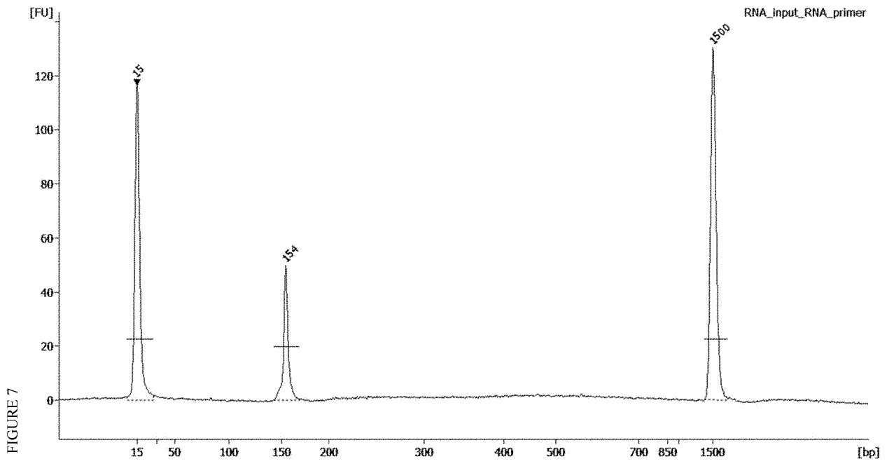

FIG. 8 shows the results from a first DNA amplification experiment. The amplification reaction mixture included the following: 5 μL 2× Platinum SuperFi RT-PCR MasterMix; 0.2 μL 10 μM DNA rev; 0.4 μL 10 μM fwd; 1.32 μL DNA; 0.5 μL Evagreen; 0.2 μL ROX; and 2.18 μL water. In this graph the Y axis shows the amount of amplification product as measured by fluorescence and the X axis shows the number of amplification cycles. In this embodiment 10 ng of DNA was used as an input. The primers utilized were THSP_PTEN_2DNA_rev_seq+THSP_PTEN_2_fwd_seq. SuperScript IV+Platinum SuperFi RT-PCR MasterMix. As each qPCR cycle amplifies target, the SYBR Green dye fluorescence is measured. The qPCR cycling parameters are displayed in the table. Once sufficient PCR amplification cycles generate an amount of amplicon product above the detection threshold, the qPCR instrument (Agilent) displays fluorescent amplification curve. When this amplification curves crosses a threshold line (Y-axis), that cycle number (X-axis) is called the threshold cycle (C_{T}).

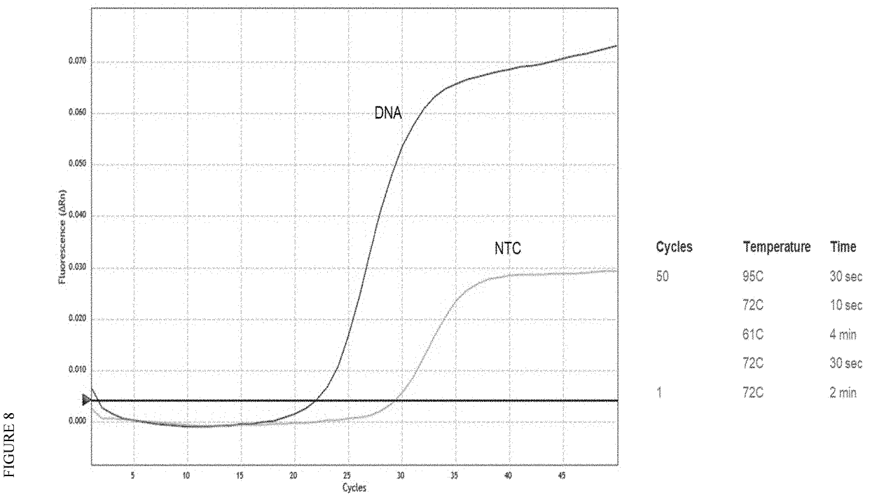

FIG. 9 shows the DNA amplification experiment of FIG. 8. The amplification reaction mixture included the following: 5μL 2× Platinum SuperFi RT-PCR MasterMix; 0.2 μL, 10 μM DNA rev; 0.4 μL 10 μM fwd; 1.32 μL DNA; 0.5 μL Evagreen; 0.2 μL ROX; and 2.18 μL water. The Y-axis shows the amount of amplification product as measured in fluorescence units, while the X axis shows the size or length of the amplicons in nucleotides. The qPCR product is analyzed on a Bioanalyzer DNA 1000 chip; 1:10 dilution. Expected THSP_PTEN_2DNA amplicon=270 bp. This Bioanalyzer displays a single PCR product from the sample of approximately 270-280 base pairs in size.

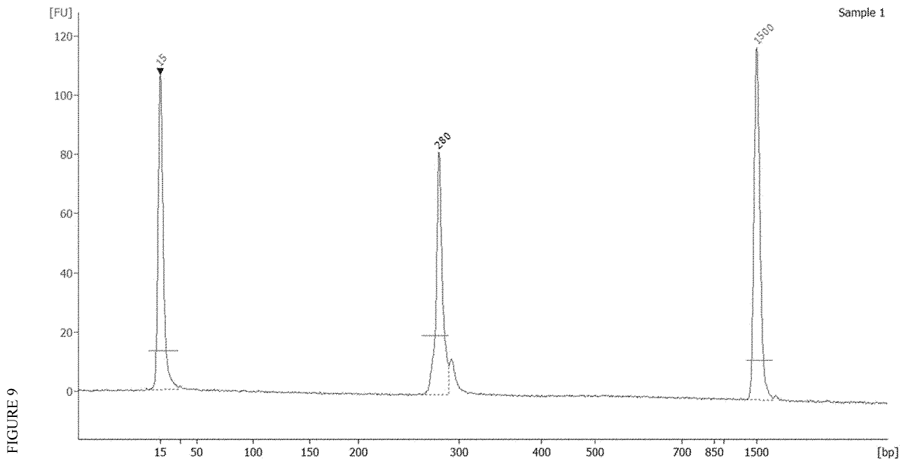

FIG. 10 shows the results from a second DNA amplification experiment. The amplification reaction mixture included the following: 5 μL 2× Platinum SuperFi RT-PCR MasterMix; 0.2 μL 10 μM DNA rev (annealed to blocking oligo); 0.4 μL 10 μM fwd; 1.32 μL DNA; 0.5 μL Evagreen; 0.2 μL ROX; and 2.18 μL water. In this graph the Y axis shows the amount of amplification product as measured by fluorescence and the X axis shows the number of amplification cycles. 10 ng of DNA was used as an input. The primers utilized were THSP_PTEN_2DNA_rev_seq+THSP_PTEN_2_fwd_seq+THSP_PTEN_2_DNA_blocking. SuperScript IV+Platinum SuperFi RT-PCR MasterMix. As each qPCR cycle amplifies target, the SYBR Green dye fluorescence is measured. The qPCR cycling parameters are displayed in the table. Once sufficient PCR amplification cycles generate an amount of amplicon product above the detection threshold, the qPCR instrument (Agilent) displays fluorescent amplification curve. When this amplification curves crosses a threshold line (Y-axis), that cycle number (X-axis) is called the threshold cycle (C_{T}).

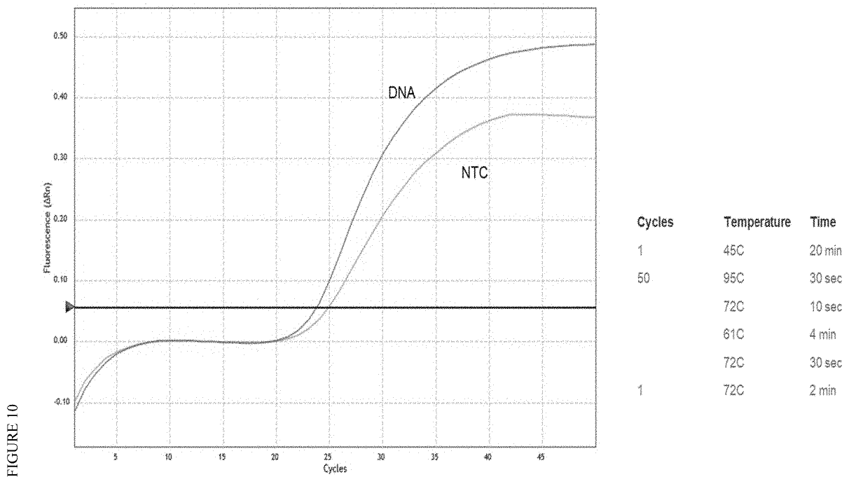

FIG. 11 shows more results from the second DNA amplification experiment shown in FIG. 10. The Y axis shows the amount of amplification product as measured in fluorescence units, while the X-axis shows the size or length of the amplicons in nucleotides. The qPCR product is analyzed on a Bioanalyzer DNA 1000 chip; 1:10 dilution. The expected THSP_PTEN_2DNA amplicon—270 bp. This Bioanalyzer displays a single PCR product from the sample of approximately 270-280 base pairs in size.

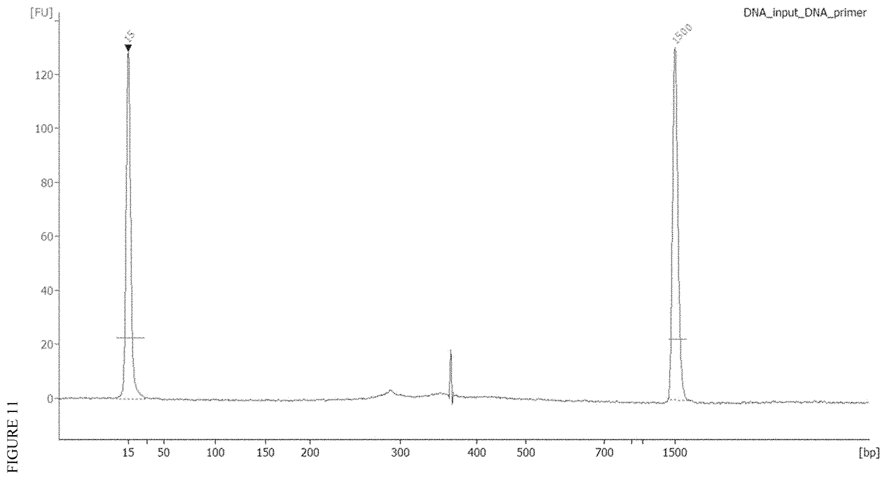

FIG. 12 shows RNA amplification using dd NTP primers. The Y axis shows the amount of amplification product as measured by fluorescence and the X axis shows the number of amplification cycles. 15 ng of RNA was used as an input. The primers utilized were THSP_PTEN_2 DNA_rev_seq_ddNTP+THSP_PTEN_2_fwd_seq_ddNTP+THSP_PTEN_2_RNA_rev. SuperScript IV+Platinum SuperFi RT-PCR MasterMix. The amplification reaction mixture included the following: 5 μL, 2× Platinum SuperFi RT-PCR MasterMix; 0.2 μL 10 μM RNA rev primer; 0.4 μL 10 μM fwd ddNTP primer; 0.2 μL 10 μM DNA rev ddNTP primer; 1.5 μL RNA; 0.25 Superscript RT; 0.5 μL Evagreen; 0.2 μL ROX; and 2.18 μL water. As each qPCR cycle amplifies target, the SYBR Green dye fluorescence is measured. The qPCR cycling parameters are displayed in the table. Once sufficient PCR amplification cycles generate an amount of amplicon product above the detection threshold, the qPCR instrument (Agilent) displays fluorescent amplification curve. When this amplification curves crosses a threshold line (Y-axis), that cycle number (X-axis) is called the threshold cycle (C_{T}).

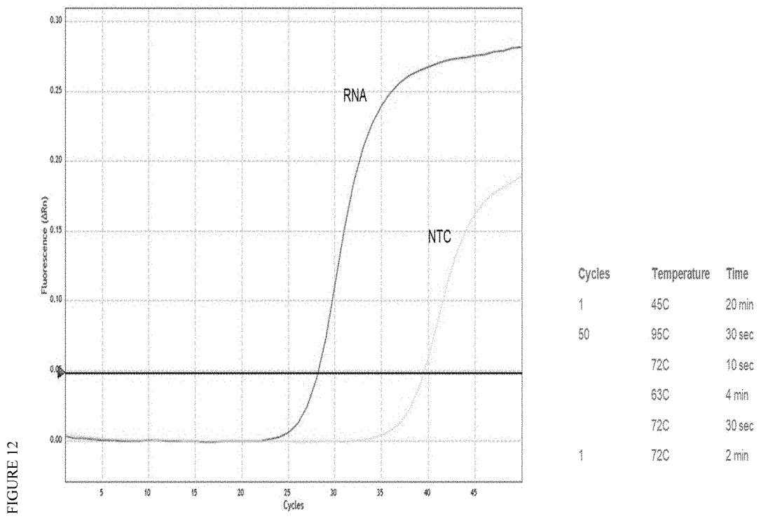

FIG. 13 shows more results from the RNA amplification using ddNTP primers depicted in FIG. 12. The amplification reaction mixture included the following: 5 μL 2× Platinum SuperFi RT-PCR MasterMix; 0.2 μL 10 μM RNA rev primer; 0.4 μL 10 μM fwd ddNTP primer; 0.2 μL 10 μM DNA rev ddNTP primer; 1.5 μl RNA; 0.25 Superscript RT; 0.5 μL Evagreen; 0.2 μL ROX; and 2.18 μL water. The Y axis shows the amount of amplification product as measured by fluorescence and the X axis shows the number of amplification cycles. The qPCR product from amplification is analyzed on a Bioanalyzer DNA 1000 chip; 1:5 dilution; Expected THSP_PTEN_2RNA amplicon=149 bp. This Bioanalyzer displays a single PCR product from the sample of approximately 149 base pairs in size.

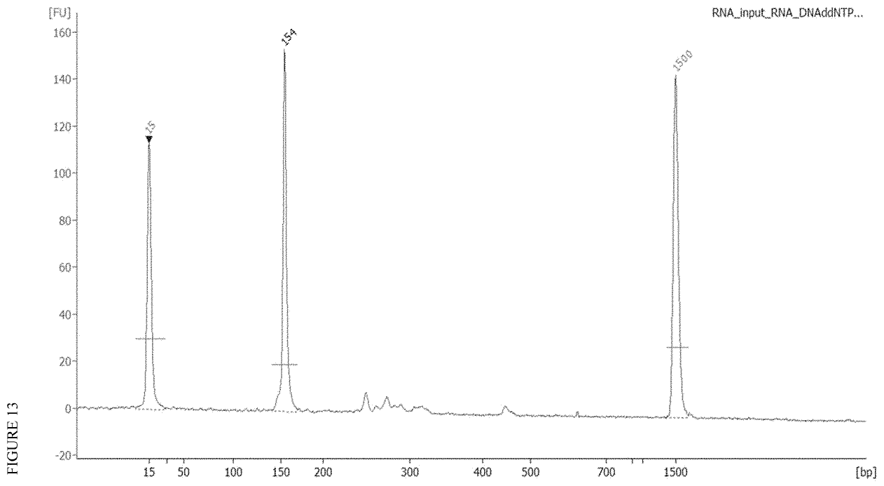

FIG. 14 shows results from a DNA amplification using ddNTP primers. The amplification reaction mixture included the following: 5 μL Platinum SuperFi RT-PCR MasterMix; 0.2 μL 10 μM RNA rev primer; 0.4 μL 10 μM fwd ddNTP primer; 0.2 μL 10 μM DNA rev ddNTP primer; 1.32 μL DNA; 0.5 μL Evagreen; 0.2 μL ROX; and 2.18 μL water. The Y axis shows the amount of amplification product as measured by fluorescence and the X axis shows the number of amplification cycles. 10 ng of DNA was used as an input. The primers utilized were THSP_PTEN_2 DNA_rev_seq ddNTP+THSP_PTEN_2_fwd_seq_ddNTP+THSP_PTEN_2_RNA_rev. SuperScript IV+Platinum SuperFi RT-PCR MasterMix. As each qPCR cycle amplifies target, the SYBR Green dye fluorescence is measured. The qPCR cycling parameters are displayed in the table. Once sufficient PCR amplification cycles generate an amount of amplicon product above the detection threshold, the qPCR instrument (Agilent) displays fluorescent amplification curve. When this amplification curves crosses a threshold line (Y-axis), that cycle number (X-axis) is called the threshold cycle (C_{T}).

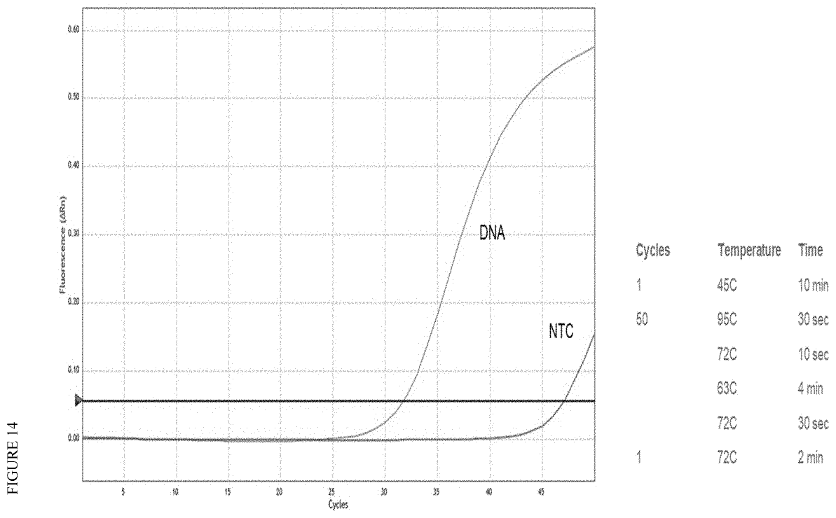

FIG. 15 shows more results from the DNA amplification using ddNTP primers depicted in FIG. 14. The amplification reaction mixture included the following: 5 μL, Platinum SuperFi RT-PCR MasterMix; 0.2 μL 10 μM RNA rev primer; 0.4 μL 10 μM fwd ddNTP primer; 0.2 μL 10 μM DNA rev ddNTP primer; 1.32 μL DNA; 0.5 μL Evagreen; 0.2 μL ROX; and 2.18 μL water. The Y axis shows the amount of amplification product as measured in fluorescence units, while the X axis shows the size or length of the amplicons in nucleotides. The qPCR product from amplification is analyzed on a Bioanalyzer DNA 1000 chip; 1:5 dilution; Expected THSP_PTEN_2 DNA amplicon=270 bp. This Bioanalyzer displays a single PCR product from the sample of approximately 270 base pairs in size.

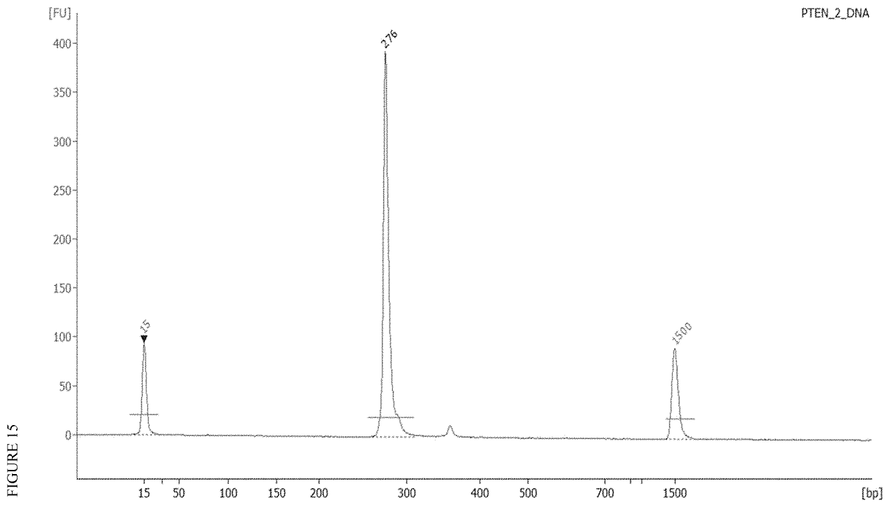

FIG. 16 shows results from an RNA+DNA amplification using ddNTP primers. The amplification reaction mixture included the following: 5 μL Superfi MasterMix; 0.2 μL 10 μM RNA rev primer; 0.4 μL 10 μM fwd ddNTP primer; 0.2 μL 10 μM DNA rev ddNTP primer; 1.5 μL RNA; 1.32 DNA; 0.25 Superscript RT; 0.5 μL Evagreen; 0.2 μL ROX; and 2.43 μL water. The Y axis shows the amount of amplification product as measured by fluorescence and the X axis shows the number of amplification cycles. 15 ng of RNA and 10 ng of DNA were used as an input. The primers used were THSP_PTEN_2 DNA_rev_seq ddNTP+THSP_PTEN_2_fwd_seq_ddNTP+THSP_PTEN_2_RNA_rev. SuperScript IV+SuperFi MasterMix. As each qPCR cycle amplifies target, the SYBR Green dye fluorescence is measured. The qPCR cycling parameters are displayed in the table. Once sufficient PCR amplification cycles generate an amount of amplicon product above the detection threshold, the qPCR instrument (Agilent) displays fluorescent amplification curve. When this amplification curves crosses a threshold line (Y-axis), that cycle number (X-axis) is called the threshold cycle (C_{T}).

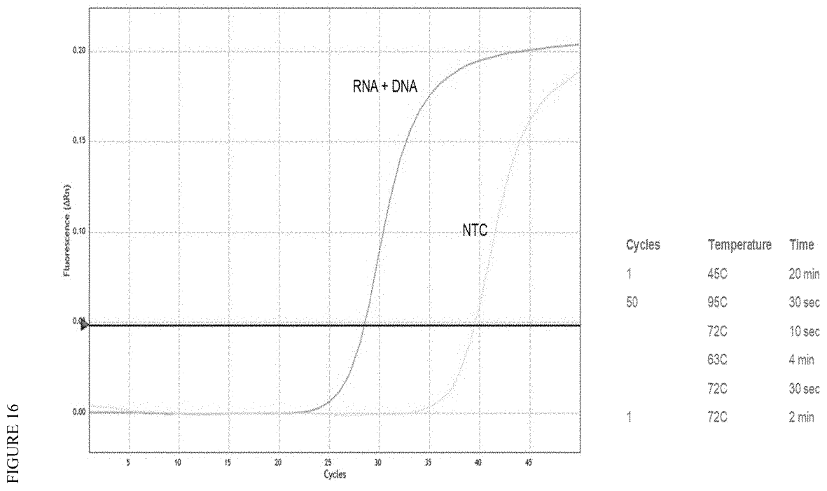

FIG. 17 shows more results from a RNA+DNA amplification using ddNTP primers shown in FIG. 16. The Y axis shows the amount of amplification product as measured in fluorescence units, while the X axis shows the size or length of the amplicons in nucleotides. The qPCR product on Bioanalyzer DNA 1000 chip. 1:5 dilution. Expected THSP_PTEN_2RNA amplicon—149 bp. Expected THSP_PTEN_2 DNA amplicon=270 bp. This Bioanalyzer displays PCR products from the sample of approximately 149-153 and 270-274 base pairs in size.

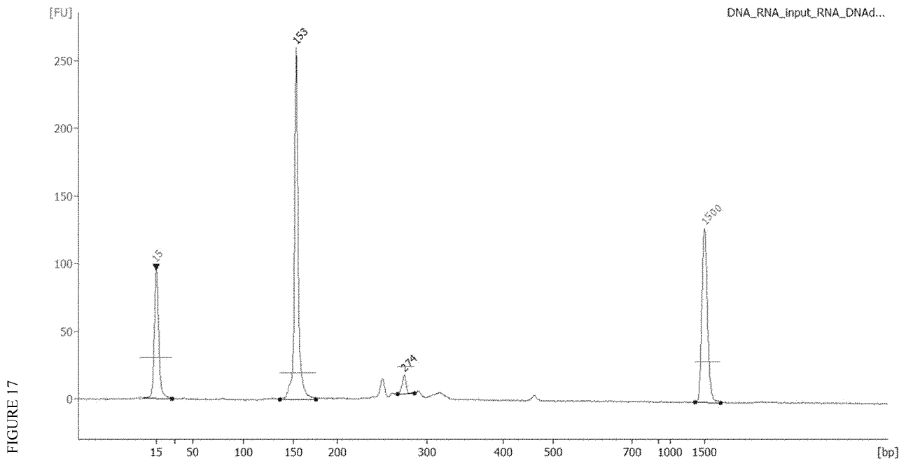

### DETAILED DESCRIPTION

Various aspects of the invention will now be described with reference to the following section which will be understood to be provided by way of illustration only and not to constitute a limitation on the scope of the invention.

“Complementarity” refers to the ability of a nucleic acid to form hydrogen bond(s) or hybridize with another nucleic acid sequence by either traditional Watson-Crick or other non-traditional types. As used herein “hybridization,” refers to the binding, duplexing, or hybridizing of a molecule only to a particular nucleotide sequence under low, medium, or highly stringent conditions, including when that sequence is present in a complex mixture (e.g., total cellular) DNA or RNA. See e.g. Ausubel, et al., Current Protocols In Molecular Biology, John Wiley & Sons, New York, N.Y., 1993. If a nucleotide at a certain position of a polynucleotide is capable of forming a Watson-Crick pairing with a nucleotide at the same position in an anti-parallel DNA or RNA strand, then the polynucleotide and the DNA or RNA molecule are complementary to each other at that position. The polynucleotide and the DNA or RNA molecule are “substantially complementary” to each other when a sufficient number of corresponding positions in each molecule are occupied by nucleotides that can hybridize or anneal with each other in order to affect the desired process. A complementary sequence is a sequence capable of annealing under stringent conditions to provide a 3′-terminal serving as the origin of synthesis of complementary chain.

“Identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as determined by the match between strings of such sequences. “Identity” and “similarity” can be readily calculated by known methods, including, but not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., Siam J. Applied Math., 48:1073 (1988). In addition, values for percentage identity can be obtained from amino acid and nucleotide sequence alignments generated using the default settings for the AlignX component of Vector NTI Suite 8.0 (Informax, Frederick, Md.). Preferred methods to determine identity are designed to give the largest match between the sequences tested. Methods to determine identity and similarity are codified in publicly available computer programs. Preferred computer program methods to determine identity and similarity between two sequences include, but are not limited to, the GCG program package (Devereux, J., et al., Nucleic Acids Research 12(1): 387 (1984)), BLASTP, BLASTN, and FASTA (Atschul, S. F. et al., J. Molec. Biol. 215:403-410 (1990)). The BLAST X program is publicly available from NCBI and other sources (BLAST Manual, Altschul, S., et al., NCBINLM NIH Bethesda, Md. 20894: Altschul, S., et al., J. Mol. Biol. 215:403-410 (1990). The well-known Smith Waterman algorithm may also be used to determine identity.

The terms “amplify”, “amplifying”, “amplification reaction”, or a “NAAT” and their variants, refer generally to any action or process whereby at least a portion of a nucleic acid molecule (referred to as a template nucleic acid molecule) is replicated or copied into at least one additional nucleic acid molecule. The additional nucleic acid molecule optionally includes sequence that is substantially identical or substantially complementary to at least some portion of the template nucleic acid molecule. The template nucleic acid molecule can be single-stranded or double-stranded and the additional nucleic acid molecule can independently be single-stranded or double-stranded. In some embodiments, amplification includes a template-dependent in vitro enzyme-catalyzed reaction for the production of at least one copy of at least some portion of the nucleic acid molecule or the production of at least one copy of a nucleic acid sequence that is complementary to at least some portion of the nucleic acid molecule. Amplification optionally includes linear or exponential replication of a nucleic acid molecule. In some embodiments, such amplification is performed using isothermal conditions; in other embodiments, such amplification can include thermocycling. In some embodiments, the amplification is a multiplex amplification that includes the simultaneous amplification of a plurality of target sequences in a single amplification reaction. At least some of the target sequences can be situated, on the same nucleic acid molecule or on different target nucleic acid molecules included in the single amplification reaction. In some embodiments, “amplification” includes amplification of at least some portion of DNA- and RNA-based nucleic acids alone, or in combination. The amplification reaction can include single or double-stranded nucleic acid substrates and can further including any of the amplification processes known to one of ordinary skill in the art. In some embodiments, the amplification reaction includes polymerase chain reaction (PCR). In the present invention, the terms “synthesis” and “amplification” of nucleic acid are used. The synthesis of nucleic acid in the present invention means the elongation or extension of nucleic acid from an oligonucleotide serving as the origin of synthesis. If not only this synthesis but also the formation of other nucleic acid and the elongation or extension reaction of this formed nucleic acid occur continuously, a series of these reactions is comprehensively called amplification. The polynucleic acid produced by the amplification technology employed is generically referred to as an “amplicon” or “amplification product.”

A number of nucleic acid polymerases can be used in the amplification reactions utilized in certain embodiments provided herein, including any enzyme that can catalyze the polymerization of nucleotides (including analogs thereof) into a nucleic acid strand. Such nucleotide polymerization can occur in a template-dependent fashion. Such polymerases can include without limitation naturally occurring polymerases and any subunits and truncations thereof, mutant polymerases, variant polymerases, recombinant, fusion or otherwise engineered polymerases, chemically modified polymerases, synthetic molecules or assemblies, and any analogs, derivatives or fragments thereof that retain the ability to catalyze such polymerization. Optionally, the polymerase can be a mutant polymerase comprising one or more mutations involving the replacement of one or more amino acids with other amino acids, the insertion or deletion of one or more amino acids from the polymerase, or the linkage of parts of two or more polymerases. Typically, the polymerase comprises one or more active sites at which nucleotide binding and/or catalysis of nucleotide polymerization can occur. Some exemplary polymerases include without limitation DNA polymerases and RNA polymerases. The term “polymerase” and its variants, as used herein, also includes fusion proteins comprising at least two portions linked to each other, where the first portion comprises a peptide that can catalyze the polymerization of nucleotides into a nucleic acid strand and is linked to a second portion that comprises a second polypeptide. In some embodiments, the second polypeptide can include a reporter enzyme or a processivity-enhancing domain. Optionally, the polymerase can possess 5′ exonuclease activity or terminal transferase activity. In some embodiments, the polymerase can be optionally reactivated, for example through the use of heat, chemicals or re-addition of new amounts of polymerase into a reaction mixture. In some embodiments, the polymerase can include a hot-start polymerase or an aptamer-based polymerase that optionally can be reactivated.

The terms “target primer” or “target-specific primer” and variations thereof refer to primers that are complementary to a binding site sequence. Target primers are generally a single stranded or double-stranded polynucleotide, typically an oligonucleotide, that includes at least one sequence that is at least partially complementary to a target nucleic acid sequence. A ‘competimer’ may have a complementary or partially complementary sequence as a target primer or target specific primer and it may incorporate modification in the nucleic acids or nucleotides. A competimer typically competes with another primer for binding to a target nucleic acid or a target nucleic acid sequence in an amplicon, and as such can enhance or select the amplification of particular amplicons in an amplification reaction. A competimer can be employed to quench specific product formation during a multiplex PCR amplification process.

“Forward primer binding site” and “reverse primer binding site” refers to the regions on the template DNA and/or the amplicon to which the forward and reverse primers bind. The primers act to delimit the region of the original template polynucleotide which is exponentially amplified during amplification. In some embodiments, additional primers may bind to the region 5′ of the forward primer and/or reverse primers. Where such additional primers are used, the forward primer binding site and/or the reverse primer binding site may encompass the binding regions of these additional primers as well as the binding regions of the primers themselves. For example, in some embodiments, the method may use one or more additional primers which bind to a region that lies 5′ of the forward and/or reverse primer binding region. Such a method was disclosed, for example, in WO0028082 which discloses the use of “displacement primers” or “outer primers”.

Barcode sequences can be incorporated into microfluidic beads to decorate the bead with identical sequence tags. Such tagged beads can be inserted into microfluidic droplets and via droplet PCR amplification, tag each target amplicon with the unique bead barcode. Such barcodes can be used to identify specific droplets upon a population of amplicons originated from. This scheme can be utilized when combining a microfluidic droplet containing single individual cell with another microfluidic droplet containing a tagged bead. Upon collection and combination of many microfluidic droplets, amplicon sequencing results allow for assignment of each product to unique microfluidic droplets. In a typical implementation, we use barcodes on the Mission Bio Tapestri beads to tag and then later identify each droplet's amplicon content. The use of barcodes is described in U.S. patent application Ser. No. 15/940,850 filed Mar. 29, 2018 by Abate, A. et al., entitled ‘Sequencing of Nucleic Acids via Barcoding in Discrete Entities’, incorporated by reference herein.

A barcode may further comprise a ‘unique identification sequence’ (UMI). A UMI is a nucleic acid having a sequence which can be used to identify and/or distinguish one or more first molecules to which the UMI is conjugated from one or more second molecules. UMIs are typically short, e.g., about 5 to 20 bases in length, and may be conjugated to one or more target molecules of interest or amplification products thereof. UMIs may be single or double stranded. In some embodiments, both a nucleic acid barcode sequence and a UMI are incorporated into a nucleic acid target molecule or an amplification product thereof. Generally, a UMI is used to distinguish between molecules of a similar type within a population or group, whereas a nucleic acid barcode sequence is used to distinguish between populations or groups of molecules. In some embodiments, where both a UMI and a nucleic acid barcode sequence are utilized, the UMI is shorter in sequence length than the nucleic acid barcode sequence.

The terms “identity” and “identical” and their variants, as used herein, when used in reference to two or more nucleic acid sequences, refer to similarity in sequence of the two or more sequences (e.g., nucleotide or polypeptide sequences). In the context of two or more homologous sequences, the percent identity or homology of the sequences or subsequences thereof indicates the percentage of all monomeric units (e.g., nucleotides or amino acids) that are the same (i.e., about 70% identity, preferably 75%, 80%, 85%, 90%, 95%, 97%, 98% or 99% identity). The percent identity can be over a specified region, when compared and aligned for maximum correspondence over a comparison window, or designated region as measured using a BLAST or BLAST 2.0 sequence comparison algorithms with default parameters described below, or by manual alignment and visual inspection. Sequences are said to be “substantially identical” when there is at least 85% identity at the amino acid level or at the nucleotide level. Preferably, the identity exists over a region that is at least about 25, 50, or 100 residues in length, or across the entire length of at least one compared sequence. A typical algorithm for determining percent sequence identity and sequence similarity are the BLAST and BLAST 2.0 algorithms, which are described in Altschul et al, Nuc. Acids Res. 25:3389-3402 (1977). Other methods include the algorithms of Smith & Waterman, Adv. Appl. Math. 2:482 (1981), and Needleman & Wunsch, J. Mol. Biol. 48:443 (1970), etc. Another indication that two nucleic acid sequences are substantially identical is that the two molecules or their complements hybridize to each other under stringent hybridization conditions.

The terms “nucleic acid,” “polynucleotides,” and “oligonucleotides” refers to biopolymers of nucleotides and, unless the context indicates otherwise, includes modified and unmodified nucleotides, and both DNA and RNA, and modified nucleic acid backbones. For example, in certain embodiments, the nucleic acid is a peptide nucleic acid (PNA) or a locked nucleic acid (LNA). Typically, the methods as described herein are performed using DNA as the nucleic acid template for amplification. However, nucleic acid whose nucleotide is replaced by an artificial derivative or modified nucleic acid from natural DNA or RNA is also included in the nucleic acid of the present invention insofar as it functions as a template for synthesis of complementary chain. The nucleic acid of the present invention is generally contained in a biological sample. The biological sample includes animal, plant or microbial tissues, cells, cultures and excretions, or extracts therefrom. In certain aspects, the biological sample includes intracellular parasitic genomic DNA or RNA such as virus or mycoplasma. The nucleic acid may be derived from nucleic acid contained in said biological sample. For example, genomic DNA, or cDNA synthesized from mRNA, or nucleic acid amplified on the basis of nucleic acid derived from the biological sample, are preferably used in the described methods. Unless denoted otherwise, whenever a oligonucleotide sequence is represented, it will be understood that the nucleotides are in 5′ to 3′ order from left to right and that “A” denotes deoxyadenosine, “C” denotes deoxycytidine, “G” denotes deoxyguanosine, “T” denotes thymidine, and “U” denotes deoxyuridine. Oligonucleotides are said to have “5′ ends” and “3′ ends” because mononucleotides are typically reacted to form oligonucleotides via attachment of the 5′ phosphate or equivalent group of one nucleotide to the 3′ hydroxyl or equivalent group of its neighboring nucleotide, optionally via a phosphodiester or other suitable linkage.

A template nucleic acid in exemplary embodiments is a nucleic acid serving as a template for synthesizing a complementary chain in a nucleic acid amplification technique. A complementary chain having a nucleotide sequence complementary to the template has a meaning as a chain corresponding to the template, but the relationship between the two is merely relative. That is, according to the methods described herein a chain synthesized as the complementary chain can function again as a template. That is, the complementary chain can become a template. In certain embodiments, the template is derived from a biological sample, e.g., plant, animal, virus, micro-organism, bacteria, fungus, etc. In certain embodiments, the animal is a mammal, e.g., a human patient. A template nucleic acid typically comprises one or more target nucleic acid. A target nucleic acid in exemplary embodiments may comprise any single or double-stranded nucleic acid sequence that can be amplified or synthesized according to the disclosure, including any nucleic acid sequence suspected or expected to be present in a sample.

Primers and oligonucleotides used in embodiments herein comprise nucleotides. A nucleotide comprises any compound, including without limitation any naturally occurring nucleotide or analog thereof, which can bind selectively to, or can be polymerized by, a polymerase. Typically, but not necessarily, selective binding of the nucleotide to the polymerase is followed by polymerization of the nucleotide into a nucleic acid strand by the polymerase; occasionally however the nucleotide may dissociate from the polymerase without becoming incorporated into the nucleic acid strand, an event referred to herein as a “non-productive” event. Such nucleotides include not only naturally occurring nucleotides but also any analogs, regardless of their structure, that can bind selectively to, or can be polymerized by, a polymerase. While naturally occurring nucleotides typically comprise base, sugar and phosphate moieties, the nucleotides of the present disclosure can include compounds lacking any one, some or all of such moieties. For example, the nucleotide can optionally include a chain of phosphorus atoms comprising three, four, five, six, seven, eight, nine, ten or more phosphorus atoms. In some embodiments, the phosphorus chain can be attached to any carbon of a sugar ring, such as the 5′ carbon. The phosphorus chain can be linked to the sugar with an intervening O or S. In one embodiment, one or more phosphorus atoms in the chain can be part of a phosphate group having P and O. In another embodiment, the phosphorus atoms in the chain can be linked together with intervening O, NH, S, methylene, substituted methylene, ethylene, substituted ethylene, CNH₂, C(O), C(CH₂), CH₂CH₂, or C(OH)CH₂R (where R can be a 4-pyridine or 1-imidazole). In one embodiment, the phosphorus atoms in the chain can have side groups having O, BH3, or S. In the phosphorus chain, a phosphorus atom with a side group other than O can be a substituted phosphate group. In the phosphorus chain, phosphorus atoms with an intervening atom other than O can be a substituted phosphate group. Some examples of nucleotide analogs are described in Xu, U.S. Pat. No. 7,405,281.

In some embodiments, the nucleotide comprises a label and referred to herein as a “labeled nucleotide”; the label of the labeled nucleotide is referred to herein as a “nucleotide label”. In some embodiments, the label can be in the form of a fluorescent moiety (e.g. dye), luminescent moiety, or the like attached to the terminal phosphate group, i.e., the phosphate group most distal from the sugar. Some examples of nucleotides that can be used in the disclosed methods and compositions include, but are not limited to, ribonucleotides, deoxyribonucleotides, modified ribonucleotides, modified deoxyribonucleotides, ribonucleotide polyphosphates, deoxyribonucleotide polyphosphates, modified ribonucleotide polyphosphates, modified deoxyribonucleotide polyphosphates, peptide nucleotides, modified peptide nucleotides, metallonucleosides, phosphonate nucleosides, and modified phosphate-sugar backbone nucleotides, analogs, derivatives, or variants of the foregoing compounds, and the like. In some embodiments, the nucleotide can comprise non-oxygen moieties such as, for example, thio- or borano-moieties, in place of the oxygen moiety bridging the alpha phosphate and the sugar of the nucleotide, or the alpha and beta phosphates of the nucleotide, or the beta and gamma phosphates of the nucleotide, or between any other two phosphates of the nucleotide, or any combination thereof. “Nucleotide 5′-triphosphate” refers to a nucleotide with a triphosphate ester group at the 5′ position, and are sometimes denoted as “NTP”, or “dNTP” and “ddNTP” to particularly point out the structural features of the ribose sugar. The triphosphate ester group can include sulfur substitutions for the various oxygens, e.g. a-thio-nucleotide 5′-triphosphates. For a review of nucleic acid chemistry, see: Shabarova, Z. and Bogdanov, A. Advanced Organic Chemistry of Nucleic Acids, VCH, New York, 1994.

Any nucleic acid amplification method may by utilized, such as a PCR-based assay, e.g., quantitative PCR (qPCR), may be used to detect the presence of certain nucleic acids, e.g., genes, of interest, present in discrete entities or one or more components thereof, e.g., cells encapsulated therein. Such assays can be applied to discrete entities within a microfluidic device or a portion thereof or any other suitable location. The conditions of such PCR-based assays may include detecting nucleic acid amplification over time and may vary in one or more ways.

The number of PCR primers that may be added to a microdroplet may vary. The number of PCR primers that may be added to a microdroplet may range from about 1 to about 500 or more, e.g., about 2 to 100 primers, about 2 to 10 primers, about 10 to 20 primers, about 20 to 30 primers, about 30 to 40 primers, about 40 to 50 primers, about 50 to 60 primers, about 60 to 70 primers, about 70 to 80 primers, about 80 to 90 primers, about 90 to 100 primers, about 100 to 150 primers, about 150 to 200 primers, about 200 to 250 primers, about 250 to 300 primers, about 300 to 350 primers, about 350 to 400 primers, about 400 to 450 primers, about 450 to 500 primers, or about 500 primers or more.

One or both primers of a primer set may comprise a barcode sequence. In some embodiments, one or both primers comprise a barcode sequence and a unique molecular identifier (UMI). In some embodiments, where both a UMI and a nucleic acid barcode sequence are utilized, the UMI is incorporated into the target nucleic acid or an amplification product thereof prior to the incorporation of the nucleic acid barcode sequence. In some embodiments, where both a UMI and a nucleic acid barcode sequence are utilized, the nucleic acid barcode sequence is incorporated into the UMI or an amplification product thereof subsequent to the incorporation of the UMI into a target nucleic acid or an amplification product thereof.

Primers may contain primers for one or more nucleic acid of interest, e.g. one or more genes of interest. The number of primers for genes of interest that are added may be from about one to 500, e.g., about 1 to 10 primers, about 10 to 20 primers, about 20 to 30 primers, about 30 to 40 primers, about 40 to 50 primers, about 50 to 60 primers, about 60 to 70 primers, about 70 to 80 primers, about 80 to 90 primers, about 90 to 100 primers, about 100 to 150 primers, about 150 to 200 primers, about 200 to 250 primers, about 250 to 300 primers, about 300 to 350 primers, about 350 to 400 primers, about 400 to 450 primers, about 450 to 500 primers, or about 500 primers or more. Primers and/or reagents may be added to a discrete entity, e.g., a microdroplet, in one step, or in more than one step. For instance, the primers may be added in two or more steps, three or more steps, four or more steps, or five or more steps. Regardless of whether the primers are added in one step or in more than one step, they may be added after the addition of a lysing agent, prior to the addition of a lysing agent, or concomitantly with the addition of a lysing agent. When added before or after the addition of a lysing agent, the PCR primers may be added in a separate step from the addition of a lysing agent. In some embodiments, the discrete entity, e.g., a microdroplet, may be subjected to a dilution step and/or enzyme inactivation step prior to the addition of the PCR reagents. Exemplary embodiments of such methods are described in PCT Publication No. WO 2014/028378, the disclosure of which is incorporated by reference herein in its entirety and for all purposes.

A primer set for the amplification of a target nucleic acid typically includes a forward primer and a reverse primer that are complementary to a target nucleic acid or the complement thereof. In some embodiments, amplification can be performed using multiple target-specific primer pairs in a single amplification reaction, wherein each primer pair includes a forward target-specific primer and a reverse target-specific primer, where each includes at least one sequence that substantially complementary or substantially identical to a corresponding target sequence in the sample, and each primer pair having a different corresponding target sequence. Accordingly, certain methods herein are used to detect or identify multiple target sequences from a single cell sample.

Primers may be designed to only selectively amplify a DNA or RNA target sequence. For example, one or both primers of a primer set may have a modification that prevent extension by a particular polymerase. For example, one or both primers of a primer set may comprise a DNA specific primer that is blocked before reverse transcriptase is added as a step in a method of detection or amplification so that cDNA is only extended by an RNA reverse primer. In another implementation, a DNA reverse primer that is outside of the RNA reverse primer is provided so that cDNA is only extended by an RNA reverse primer.

In one embodiment, the target nucleic acid may comprise both DNA and RNA, and either DNA or RNA is selectively amplified to form an amplicon product specific for either a DNA or an RNA target nucleic acid. In certain implementations of embodiments of the invention, DNA or RNA amplicons are attenuated, limited, or prevented during amplification. Some embodiments use competimers that selectively modify the amplification of DNA or RNA amplicons. Other embodiments use biotinylated primers that selectively amplify DNA or RNA amplicons. In certain implementations of embodiments of the invention, a portion of amplification primers provided for RNA amplification comprise uracil and enable the removal RNA amplicons by cleavage.

A number of approaches may be utilized to block the extension of particular primers, for example during a particular part of a reaction. These include modifications, spacers, and other non-natural oligonucleotide primers. In certain implements, blocking oligos are the reverse compliment of the DNA reverse primer GSP region with/3SpC3/to block any extension.

In certain implements, ddNTP mismatch primers are the forward primers and DNA reverse primers with /3ddC/ added to block extension until the Hotstart polymerase is activated. If a C following the primer is not a mismatch, A/3ddC/ was added. These ddNTP mismatch primers were tested and analyzed on a ThermoFisher Multiple Primer analysis along with the RNA reverse primers to confirm no primer interactions where the hotstart polymerase could repair during the reverse transcription if it retains 3′ to 5′ exonuclease activity at room temperature. Dideoxy C is the only dideoxy IDT has available. Alternate embodiments utilize TdTon ddNTPsin 4 pools.

Some of the exemplary primer sets developed according to methods of the invention are shown in the Tables below.

TABLE I
Primer set developed for feasibility studies showing gene specific portions

| Amp ID | fwd_seq | DNA_rev_seq | RNA_rev_seq |
|---|---|---|---|
| THSP_HRAS_1 | GGATGTCCTCAAAAGACT | GGAAGCAGGTGGTCATTGAT | CCTGTTGGACATC (SEQ ID |
| | TGGTGT (SEQ ID NO: 4) | GG (SEQ ID NO: 16) | NO: 6) |
| THSP_MET_5 | GTCTTTCCCCACAATCATA | AAACCATCTTTCGTTTCCTTTA | CAATGGATGATCTG (SEQ ID |
| | CTGCT (SEQ ID NO: 8) | GCC (SEQ ID NO: 17) | NO: 26) |
| THSP_FGFR3_1 | GCGCCTTTCGAGCAGTAC | GCTCTGTGTAGCTGTCTCTCC | CTGCACTGAGTCT (SEQ ID |
| | TC (SEQ ID NO: 9) | A (SEQ ID NO: 18) | NO: 27) |
| THSP_NOTCH1_1 | AGACGTTGGAATGCGGGG | GCACACTATTCTGCCCCAGG | ATCCTCGCTGGT (SEQ ID |
| | AC (SEQ ID NO: 10) | A (SEQ ID NO: 19) | NO: 28) |
| THSP_PIK3CA_12 | TTCTCAATGATGCTTGGCT | CATGCTGTTTAATTGTGTGGA | CACCATGATGTGC (SEQ ID |
| | CTG (SEQ ID NO: 11) | AGAT (SEQ ID NO: 20) | NO: 7) |
| THSP_TP53_1 | GGCTGTCCCAGAATGCAA | CAAGCAATGGATGATTTGATG | GAACAATGGTTCACT (SEQ |
| | GAAG (SEQ ID NO: 12) | CTGT (SEQ ID NO: 21) | ID NO: 29) |
| THSP_PTEN_2 | GTAAATACATTCTTCATAC | AACTGACCTTAAAATTTGGAG | GCAAATGCTATCG (SEQ ID |
| | CAGGACCAGAG (SEQ ID | AAAAGTATC (SEQ ID NO: 22) | NO: 30) |
| | NO: 3) | | |
| THSP_MET_3 | ATCTGTTGTACCACTCCTT | TCCAGTACATTTTCATTGCCC | CATCACTGGCTTT (SEQ ID |
| | CCCT (SEQ ID NO: 13) | ATTG (SEQ ID NO: 23) | NO: 31) |
| THSP_APC_4 | AGCGAAGTTCCAGCAGTG | AGCTGGCAATCGAACGACTC | CTATCAAGTGAACTG (SEQ |
| | TC (SEQ ID NO: 14) | (SEQ ID NO: 24) | ID NO: 32) |
| THSP_APC_5 | ACAGAGTAGAAGTGGTCA | AGCTGACCTAGTTCCAATCTT | TTTGCAGGGTATTA (SEQ ID |
| | GCC (SEQ ID NO: 15) | TTC (SEQ ID NO: 25) | NO: 5) |

TABLE 2
RNA reverse primers.

| | Gene Specific | | | Gene specific with tail | | |
|---|---|---|---|---|---|---|
| | Tm | GC | Length | Tm | GC | Length |
| Amp ID | (C.) | (%) | (nt) | (C.) | (%) | (nt) |
| THSP_HRAS_1 | 47.9 | 58.3 | 13 | 76.1 | 53.2 | 47 |
| THSP_MET_5 | 46.1 | 42.9 | 14 | 75.3 | 50.0 | 48 |
| THSP_FGFR3_1 | 49.6 | 53.8 | 13 | 76.7 | 53.2 | 47 |
| THSP_NOTCH1_1 | 50.7 | 58.3 | 12 | 76.7 | 54.3 | 46 |
| THSP_PIK3CA_12 | 49.5 | 53.8 | 13 | 76.4 | 53.2 | 47 |
| THSP_TP53_1 | 50.4 | 40.0 | 15 | 70.6 | 49.0 | 49 |
| THSP_PTEN_2 | 46.7 | 46.2 | 13 | 76.5 | 51.1 | 47 |
| THSP_MET_3 | 47.8 | 46.2 | 13 | 76.5 | 51.1 | 47 |
| THSP_APC_4 | 47.0 | 40.0 | 15 | 75.0 | 49.0 | 49 |
| THSP_APC_5 | 47.0 | 35.7 | 14 | 75.3 | 47.9 | 48 |

TABLE 3A
Primer Sequences

| AmpID | fwd_seq | DNA_rev_seq |
|---|---|---|
| THSP_HRAS_1 | AAGCAGTGGTATCAACGCAGAGTAG | GTCTCGTGGGCTCGGAGATGTGTATAAGAGACA |
| | GGATGTCCTCAAAAGACTTGGTGT | GGGAAGCAGGTGGTCATTGATGG (SEQ ID NO: |
| | (SEQ ID NO: 33) | 43) |
| THSP_MET_5 | AAGCAGTGGTATCAACGCAGAGTAG | GTCTCGTGGGCTCGGAGATGTGTATAAGAGACA |
| | GTCTTTCCCCACAATCATACTGCT | GAAACCATCTTTCGTTTCCTTTAGCC (SEQ ID NO: |
| | (SEQ ID NO: 34) | 44) |
| THSP_FGFR3_1 | AAGCAGTGGTATCAACGCAGAGTAG | GTCTCGTGGGCTCGGAGATGTGTATAAGAGACA |
| | GCGCCTTTCGAGCAGTACTC (SEQ ID | GGCTCTGTGTAGCTGTCTCTCCA (SEQ ID NO: 45) |
| | NO: 35) | |
| THSP_NOTCH1_ | AAGCAGTGGTATCAACGCAGAGTAG | GTCTCGTGGGCTCGGAGATGTGTATAAGAGACA |
| 1 | AGACGTTGGAATGCGGGGAC (SEQ | GGCACACTATTCTGCCCCAGGA (SEQ ID NO: 46) |
| | ID NO: 36) | |
| THSP_PIK3CA_ | AAGCAGTGGTATCAACGCAGAGTAG | GTCTCGTGGGCTCGGAGATGTGTATAAGAGACA |
| 12 | TTCTCAATGATGCTTGGCTCTG (SEQ | GCATGCTGTTTAATTGTGTGGAAGAT (SEQ ID |
| | ID NO: 37) | NO: 47) |
| THSP_TP53_1 | AAGCAGTGGTATCAACGCAGAGTAG | GTCTCGTGGGCTCGGAGATGTGTATAAGAGACA |
| | GGCTGTCCCAGAATGCAAGAAG | GCAAGCAATGGATGATTTGATGCTGT (SEQ ID |
| | (SEQ ID NO: 38) | NO: 48) |
| THSP_PTEN_2 | AAGCAGTGGTATCAACGCAGAGTAG | GTCTCGTGGGCTCGGAGATGTGTATAAGAGACA |
| | GTAAATACATTCTTCATACCAGGACC | GAACTGACCTTAAAATTTGGAGAAAAGTATC |
| | AGAG (SEQ ID NO: 39) | (SEQ ID NO: 49) |
| THSP_MET_3 | AAGCAGTGGTATCAACGCAGAGTAG | GTCTCGTGGGCTCGGAGATGTGTATAAGAGACA |
| | ATCTGTTGTACCACTCCTTCCCT | GTCCAGTACATTTTCATTGCCCATTG (SEQ ID NO: |
| | (SEQ ID NO: 40) | 50) |
| THSP_APC_4 | AAGCAGTGGTATCAACGCAGAGTAG | GTCTCGTGGGCTCGGAGATGTGTATAAGAGACA |
| | AGCGAAGTTCCAGCAGTGTC (SEQ ID | GAGCTGGCAATCGAACGACTC (SEQ ID NO: 51) |
| | NO: 41) | |
| THSP_APC_5 | AAGCAGTGGTATCAACGCAGAGTAG | GTCTCGTGGGCTCGGAGATGTGTATAAGAGACA |
| | ACAGAGTAGAAGTGGTCAGCC (SEQ | GAGCTGACCTAGTTCCAATCTTTTC (SEQ ID NO: |
| | ID NO: 42) | 52) |

TABLE 3B
Primer Sequences

| AmpID | RNA_rev_seq | RNA_rev_seq_2nt | DNA_blocking |
|---|---|---|---|
| THSP_HRAS_ | GTCTCGTGGGCTCGGAGATGTG | GTCTCGTGGGCTCGGAGATGT | CCATCAATGACCACCT |
| 1 | TATAAGAGACAGCCTGTTGGACA | GTATAAGAGACAGTGTTGGACA | GCTTCC/3SpC3/ (SEQ |
| | TC (SEQ ID NO: 53) | TC (SEQ ID NO: 63) | ID NO: 73) |
| THSP_MET_ | GTCTCGTGGGCTCGGAGATGTG | GTCTCGTGGGCTCGGAGATGT | GGCTAAAGGAAACGA |
| 5 | TATAAGAGACAGCAATGGATGAT | GTATAAGAGACAGATGGATGAT | AAGATGGTTT/3SpC3/ |
| | CTG (SEQ ID NO: 54) | CTG (SEQ ID NO: 64) | (SEQ ID NO: 74) |
| THSP_ | GTCTCGTGGGCTCGGAGATGTG | GTCTCGTGGGCTCGGAGATGT | TGGAGAGACAGCTAC |
| FGFR3_1 | TATAAGAGACAGCTGCACTGAGT | GTATAAGAGACAGGCACTGAGT | ACAGAGC/3SpC3/ |
| | CT (SEQ ID NO: 55) | CT (SEQ ID NO: 65) | (SEQ ID NO: 75) |
| THSP_ | GTCTCGTGGGCTCGGAGATGTG | GTCTCGTGGGCTCGGAGATGT | TCCTGGGGCAGAATA |
| NOTCH1_ | TATAAGAGACAGATCCTCGCTGG | GTATAAGAGACAGCCTCGCTGG | GTGTGC/3SpC3/ (SEQ |
| 1 | T (SEQ ID NO: 56) | T (SEQ ID NO: 66) | ID NO: 76) |
| THSP_ | GTCTCGTGGGCTCGGAGATGTG | GTCTCGTGGGCTCGGAGATGT | ATCTTCCACACAATTA |
| PIK3CA_ | TATAAGAGACAGCACCATGATGT | GTATAAGAGACAGCCATGATGT | AACAGCATG/3SpC3/ |
| 12 | GC (SEQ ID NO: 57) | GC (SEQ ID NO: 67) | (SEQ ID NO: 77) |
| THSP_TP53_ | GTCTCGTGGGCTCGGAGATGTG | GTCTCGTGGGCTCGGAGATGT | ACAGCATCAAATCATC |
| 1 | TATAAGAGACAGGAACAATGGTT | GTATAAGAGACAGACAATGGTT | CATTGCTTG/3SpC3/ |
| | CACT (SEQ ID NO: 58) | CACT (SEQ ID NO: 68) | (SEQ ID NO: 78) |
| THSP_PTEN_ | GTCTCGTGGGCTCGGAGATGTG | GTCTCGTGGGCTCGGAGATGT | GATACTTTTCTCCAAA |
| 2 | TATAAGAGACAGGCAAATGCTAT | GTATAAGAGACAGAAATGCTAT | TTTTAAGGTCAGTT/ |
| | CG (SEQ ID NO: 59) | CG (SEQ ID NO: 69) | 3SpC3/ (SEQ ID NO: 79) |
| THSP_MET_ | GTCTCGTGGGCTCGGAGATGTG | GTCTCGTGGGCTCGGAGATGT | CAATGGGCAATGAAAA |
| 3 | TATAAGAGACAGCATCACTGGCT | GTATAAGAGACAGTCACTGGCT | TGTACTGGA/3SpC3/ |
| | TT (SEQ ID NO: 60) | TT (SEQ ID NO: 70) | (SEQ ID NO: 80) |
| THSP_APC_ | GTCTCGTGGGCTCGGAGATGTG | GTCTCGTGGGCTCGGAGATGT | GAGTCGTTCGATTGCC |
| 4 | TATAAGAGACAGCTATCAAGTGA | GTATAAGAGACAGATCAAGTGA | AGCT/3SpC3/ (SEQ ID |
| | ACTG (SEQ ID NO: 61) | ACTG (SEQ ID NO: 71) | NO: 81) |
| THSP_APC_ | GTCTCGTGGGCTCGGAGATGTG | GTCTCGTGGGCTCGGAGATGT | GAAAAGATTGGAACTA |
| 5 | TATAAGAGACAGTTTGCAGGGTA | GTATAAGAGACAGTGCAGGGTA | GGTCAGCT/3SpC3/ |
| | TTA (SEQ ID NO: 62) | TTA (SEQ ID NO: 72) | (SEQ ID NO: 82) |

TABLE 4
Primer Sequences

| AmpID | fwd_seq_ddNTP | DNA_rev_seq_ddNTP |
|---|---|---|
| THSP_HRAS_1 | AAGCAGTGGTATCAACGCAGAGT | GTCTCGTGGGCTCGGAGATGTGTATAAGAGACA |
| | AGGGATGTCCTCAAAAGACTTGG | GGGAAGCAGGTGGTCATTGATGG/3ddC/ (SEQ ID |
| | TGT/3ddC/ (SEQ ID NO: 83) | NO: 93) |
| THSP_MET_5 | AAGCAGTGGTATCAACGCAGAGT | GTCTCGTGGGCTCGGAGATGTGTATAAGAGACA |
| | AGGTCTTTCCCCACAATCATACT | GAAACCATCTTTCGTTTCCTTTAGCC/3ddC/ (SEQ |
| | GCT/3ddC/ (SEQ ID NO: 84) | ID NO: 94) |
| THSP_FGFR3_1 | AAGCAGTGGTATCAACGCAGAGT | GTCTCGTGGGCTCGGAGATGTGTATAAGAGACA |
| | AGGCGCCTTTCGAGCAGTACTCA/ | GGCTCTGTGTAGCTGTCTCTCCA/3ddC/ (SEQ ID |
| | 3ddC/ (SEQ ID NO: 85) | NO: 95) |
| THSP_NOTCH1_1 | AAGCAGTGGTATCAACGCAGAGT | GTCTCGTGGGCTCGGAGATGTGTATAAGAGACA |
| | AGAGACGTTGGAATGCGGGGAC/ | GGCACACTATTCTGCCCCAGGA/3ddC/ (SEQ ID |
| | 3ddC/ (SEQ ID NO: 86) | NO: 96) |
| THSP_PIK3CA_ | AAGCAGTGGTATCAACGCAGAGT | GTCTCGTGGGCTCGGAGATGTGTATAAGAGACA |
| 12 | AGTTCTCAATGATGCTTGGCTCTG/ | GCATGCTGTTTAATTGTGTGGAAGATA/3ddC/ |
| | 3ddC/ (SEQ ID NO: 87) | (SEQ ID NO: 97) |
| THSP_TP53_1 | AAGCAGTGGTATCAACGCAGAGT | GTCTCGTGGGCTCGGAGATGTGTATAAGAGACA |
| | AGGGCTGTCCCAGAATGCAAGAAGA/ | GCAAGCAATGGATGATTTGATGCTGTA/3ddC/ |
| | 3ddC/ (SEQ ID NO: 88) | (SEQ ID NO: 98) |
| THSP_PTEN_2 | AAGCAGTGGTATCAACGCAGAGT | GTCTCGTGGGCTCGGAGATGTGTATAAGAGACA |
| | AGGTAAATACATTCTTCATACCAG | GAACTGACCTTAAAATTTGGAGAAAAGTATC/3ddC/ |
| | GACCAGAG/3ddC/ (SEQ ID NO: | (SEQ ID NO: 99) |
| | 89) | |
| THSP_MET_3 | AAGCAGTGGTATCAACGCAGAGT | GTCTCGTGGGCTCGGAGATGTGTATAAGAGACA |
| | AGATCTGTTGTACCACTCCTTCC | GTCCAGTACATTTTCATTGCCCATTG/3ddC/ (SEQ |
| | CT/3ddC/ (SEQ ID NO: 90) | ID NO: 100) |
| THSP_APC_4 | AAGCAGTGGTATCAACGCAGAGT | GTCTCGTGGGCTCGGAGATGTGTATAAGAGACA |
| | AGAGCGAAGTTCCAGCAGTGTC/ | GAGCTGGCAATCGAACGACTC/3ddC/ (SEQ ID |
| | 3ddC/ (SEQ ID NO: 91) | NO: 101) |
| THSP_APC_5 | AAGCAGTGGTATCAACGCAGAGT | GTCTCGTGGGCTCGGAGATGTGTATAAGAGACA |
| | AGACAGAGTAGAAGTGGTCAGCC/ | GAGCTGACCTAGTTCCAATCTTTTC/3ddC/ (SEQ |
| | 3ddC/ (SEQ ID NO: 92) | ID NO: 102) |

Other aspects of the invention may be described in the follow exemplary embodiments:

1. A composition or system for performing a method described herein.

2. A composition or system according to embodiment 1 comprising one or more nucleic acid amplification primer sets, wherein each primer set is complementary to a target nucleic acid and at least one primer of a nucleic acid amplification primer set comprises a barcode identification sequence.

3. A composition or system according to embodiment 1 comprising an affinity reagent that comprises a nucleic acid sequence complementary to the identification barcode sequence of one of more nucleic acid primer of a primer set, wherein said affinity reagent comprising said nucleic acid sequence complementary to the identification barcode sequence is capable of binding to a nucleic acid amplification primer set comprising a barcode identification sequence.

4. A transcriptome library generated according to a method described herein.

5. A genomic and transcriptome library generated according to a method described herein.

6. A kit or apparatus for performing a method described herein.

7. A system for performing a method described herein.

8. A composition or system according to embodiment 1, wherein the target nucleic acid is either DNA or RNA.

9. A composition or system according to embodiment 1, wherein both DNA and RNA amplification products are produced from the target nucleic acid sequence.

10. A composition or system according to embodiment 1, further comprising a reverse transcriptase polymerase.

11. A composition or system according to embodiment 1, further comprising a DNA reverse primer that is blocked.

12. A composition or system according to embodiment 1, comprising a DNA reverse primer that is outside of the RNA reverse primer.

13. A composition or system according to embodiment 1, comprising competimers that selectively modulate DNA or RNA amplicon amplification.

14. A composition or system according to embodiment 1, comprising biotinylated primers that selectively amplify DNA or RNA amplicons.

15. A composition or system according to embodiment 1, wherein a portion of library primers provided for RNA amplification comprise uracil and enable the removal RNA amplicons by cleavage.

16. A composition or system according to embodiment 1, wherein each primer set comprises a forward primer and a reverse primer that are complementary to a target nucleic acid or the complement thereof.

The following Examples are included for illustration and not limitation.

### EXAMPLE 1

### Primer Design

RNA reverse primers for 10 existing tumor hotspot panel amplicons were initially designed, choosing genes expressed in the Universal Human Reference RNA. The corresponding forward primers and DNA reverse primers, forward primers and DNA reverse primers with ddNTP mismatches at the 3′ end, blocking oligos for the DNA forward primers, and other primers were obtained from. qPCR assays were performed to determine the amplification efficiency of these primers with SYBR or EvaGreen. Universal Human Reference RNA was obtained from Agilent (Santa Clara, Calif.) and Promega Male DNA were obtained from Promega (Madison, Wis.) to perform these assays in bulk. Our templates used included RNA, DNA, and RNA+DNA (ratio of 10 to 6.6) all with annealing temperatures of 60 C.

The reverse transcription was performed off instrument, and then the samples were amplified on the qPCR instrument (Agilent, Santa Clara, Calif.). We observed Ct measurements back to the reported gene expression for the Universal Human Reference RNA. Reverse transcription was initially started with SuperScript, and then adding an aliquot to the barcoding reaction with Platinum HiFi Taq. Once feasibility was demonstrated, we tested WarmStart Rtx for the RT and Kapa2G or other multiplex high-fidelity polymerases as well as RT-PCR mastermixes such as the SuperScript IV One-Step RT-PCR System. This assay was used to optimize buffer compositions, incorporating the expected volume of cell lysis buffer, prior to testing on single cells.

| RT - Superscript III | | | final | |
|---|---|---|---|---|
| 3 | uL | RNA | 3 | ng |
| 1 | uL | 10 mM each dNTPs | 0.5 | mM each |
| 1 | uL | 2 uM GSP | 2 | pmol |
| 8 | | dH2O | | |

heat to 65 C. for 5 min, ice 1 min

| 1 | uL | 100 mM DTT | 5 | mM |
|---|---|---|---|---|
| 4 | uL | 5× first strand buffer | 1× | |
| 1 | uL | Superscript III | 200 | units |

45 C. (55 C. recommended for GSP) 30-60 min, 70 C. 15 min

| qPCR with Platinum HiFi Taq | | | final | |
|---|---|---|---|---|
| 1 | uL | 10× HiFi Buffer | 1× | |
| 0.4 | uL | 50 mM MgSO4 | 2.0 | mM |
| 0.2 | uL | 10 mM each dNTP | 0.2 | mM |
| 1 | uL | 2 uM fwd | 200 | nM each |
| 1 | uL | 2 uM rev | 200 | nM each |
| 5 | uL | cDNA | 1 | ng RNA |
| 0.4 | uL | Platinum Taq HiFi | 50 | units |

| | | | |
|---|---|---|---|
| 0.4 | uL | ROX | |
| 0.5 | uL | 20× EvaGreen | 1× |
| 0.1 | uL | dH2O | |

| | | | | |
|---|---|---|---|---|
| 40 cycles | 99 | C. | 2 | min |
| | 99 | C. | 15 | sec |
| | 60 | C. | 4 | min |

| | | |
|---|---|---|
| 4 | C. | hold |

In this embodiment, a RNA reverse primer is designed to prime at or below about 45° C. This will allow gene specific priming at the temperatures required for reverse transcription and would minimize gene specific genomic DNA priming during the higher annealing temperatures used during barcoding PCR, Because the RNA reverse primer has the barcode sequencing adaptor (PCR handle) tail found on all of the reverse primers, it is able to prime the cDNA at the higher barcoding PCR annealing temperature, but not the gDNA present in the emulsion.

For this embodiment, amplicons from the NMI, (tumor hotspot panel) were used. The entire DNA amplicon is within an exon. In this embodiment, the same forward primer design and DNA reverse primer designs were used for the DNA amplicons. The RNA amplicon may also use the same forward primer. The RNA reverse primers were designed using the IDT PrimerQuest tool inputting the DNA amplicon with the DNA reverse primer trimmed. This is to amplify the same region as the DNA amplicon but the DNA reverse primer would not be able to amplify the cDNA during the barcoding PCR cycles. The Tm parameters used in PrimerQuest were 45 C minimum, 45 C optimal, and 50 C maximum. The minimum length was lowered to 12 nts with an optimal of 20 nts and also chose the targeted region to be within the last 40 bases of the input. We also designed primers where we trimmed −2 to 4 bases off the 5′ end of these designs to lower the Tm below what PrimerQuest allows.

The secondary structures of potential RNA reverse primers were viewed with the IDT hairpin tool and it was confirmed that there were no problematic secondary structures. These primers were then blast against the human genome and transcriptome using NCBI blast to verify that the expected gene or transcript was listed.

Once a potential RNA reverse primer was chosen for a target, the primer pair of the RNA reverse primer and forward primer was input into the University of Manchester SNPcheck3 to confirm there are no expected SNVs in the general population that could affect hybridization or extension. Any primers with a SNP within the last 4 bases of the 3′ end were redesigned.

The RNA reverse and forward primers were then input into the NCBI Primer Blast tool to determine any off-target effects. Any primer set that had off target amplicons with lengths that could compete with the expected product or without mismatches were redesigned.

The full set of primers with their tails were also input into the Thermo Fisher Multiple Primer Analyzer tool to confirm no priming should occur off the tail sequences.

Some embodiments are further directed at minimizing off target effects and primer interactions. In these embodiments, a blocking oligo can be used to inhibit the DNA reverse polymerase from hybridizing during the reverse transcription either synthesizing cDNA or creating primer artifacts. These blocking oligos may he designed to hybridize to the gene specific primer portion of the DNA reverse primer and will have a 3′ C3 spacer. Because this gene specific priming region has a Tm of about 60 C, the blocking oligo may not denature during the reverse transcription.

In other embodiments, the 3′-5′ exonuclease activity of high-fidelity polymerases was used to avoid any extension of the DNA reverse primers and forward primers during the reverse transcription. The DNA reverse primers and forward primers obtained with a mismatched ddNTP on the 3′ end. Each forward primer and DNA reverse primer were ordered with a dideoxy C unless that would match the first base of the insert. In those cases, an. A was added prior to the dideoxy C.

An exemplary embodiment of a method for the design of RNA primers includes the following general processes and steps:

a. Choose amplicons from Tumor Hotspot panel where the DNA primers will amplify RNA

b. Take the amplicon sequence from the Tumor Hotspot Panel, remove the DNA reverse primer sequence and use IDT Primer Quest to design RNA reverse primers

c. Select primers with a Tm of about 45 C-50C, with 45 C optimal in some embodiments

d. Select primers with a length of about 12-30 nts, with 20 nts optimal in some embodiments

e. Use NCBI blast to verify the RNA primer sequence has the expected gene listed

f. Use IDT hairpin tool to make sure no secondary structures

g. Use Univ of Manchester SNPcheck3 to confirm no SNPs within 5 bases of the 3′ end of the reverse primer, if possible.

h. Use Thermo Fisher Multiple Primer Analyzer to predict primer interactions

i. Use NCBI Primer Blast to verify specificity of each primer pair

j. Add tails and recheck secondary structure with IDT hairpin tool and primer interactions with Thermo Fisher Multiple Primer Analysis. Secondary structures with the RNA reverse primers preferably have a Tm<50C in PCR salt conditions.

### Example II

### Polymerase Exonuclease Activity and Extension Blocking Experiments

A high fidelity polymerase, following hotstart, was tested to determine if it can remove the ddNTP mismatch once the primers have hybridized. The reverse transcriptase does not possess 3′-5′ exonuclease activity to repair these oligos during the lower temperature reaction. Any primer interactions during the lower temperature reaction would denature along with the gDNA during the hotstart.

The DNA primers were tested in the presence of RNA, DNA, and DNA and RNA. With the Platinum. SuperFi DNA polymerase, we observed the expected DNA amplicon with. DNA and also DNA and. RNA as the input. The SuperFi polymerase was able to remove the ddCTP on both primers and continue nucleotide incorporation to produce the expected amplicon. With. Platinum Taq DNA Polymerase High Fidelity, using conditions that produce a DNA amplicon with traditional primers, no DNA amplicon is observed when primers with ddNTPs are used. Blocked. DNA reverse primers and blocked forward primers were also tested with the RNA reverse primer in the presence of RNA, DNA, and DNA and RNA. Using the SuperScript IV Otte-Step RT-PCR System for the reverse transcription and PCR, we observed the expected RNA amplicon in the presence of RNA, the expected DNA amplicon in the presence of DNA, both expected DNA and RNA amplicons in the presence of DNA and RNA, and neither amplicon in the NTC.

In another experiment, representing another embodiment, the extension of the DNA primers was blocked during reverse transcription with 3-O-nitrolbenzyl on the 3′ end of the DNA reverse primer. This moiety is photocleavable and can be removed during the UV step in the workflow. Reverse transcription may be performed prior to the UV treatment then follow with the barcoding PCR. 3-O-nitrobnezy dATP is commercially available

In another experiment, representing another embodiment, blocking the extension of DNA primers is tested during reverse transcription with a 3-O-nitrolbenzyl on the 3′ end of the DNA reverse primer. This moiety is photocleavable and can be removed during the UV cleavage step in the workflow. In this embodiment, the DNA reverse primers can be tested with this 3′ photocleavable moiety and perform reverse transcription followed by UV treatment then followed with barcoding PCR. DNA amplicon would be expected in this embodiment when the UV treatment is used and no product when there is no UV cleavage performed.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “consisting essentially of”, and “consisting of” may be replaced with either of the other two terms in the specification. Also, the terms “comprising”, “including”, “containing”, etc. are to be read expansively and without limitation. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

What is claimed is:

1. A method for detection of a target nucleic acid from a single cell, the method comprising, independent of order presented, the following:
i) selecting one or more target nucleic acid sequence of interest in an individual cell, wherein the target nucleic acid sequence is complementary to a nucleic acid in a cell;
ii) providing a sample having a plurality of individual single cells; encapsulating one or more individual cell(s) in a reaction mixture comprising a protease;
iii) incubating the encapsulated cell with the protease in the drop to produce a cell lysate;
iv) providing one or more nucleic acid amplification primer sets, wherein each primer set is complementary to a target nucleic acid and at least one primer of a nucleic acid amplification primer set comprises a barcode identification sequence;
v) performing a nucleic acid amplification reaction to form an amplification product from the nucleic acid of a single cell, said amplification product comprising amplicons of one or more target nucleic acid sequence;
vi) providing an affinity reagent that comprises a nucleic acid sequence complementary to the identification barcode sequence of one of more nucleic acid primer of a primer set, wherein said affinity reagent comprising said nucleic acid sequence complementary to the identification barcode sequence is capable of binding to a nucleic acid amplification primer set comprising a barcode identification sequence;
vii) contacting an affinity reagent to the amplification product comprising amplicons of one or more target nucleic acid sequence under conditions sufficient for binding of the affinity reagent to the target nucleic acid to form an affinity reagent bound target nucleic acid; and
viii) determining the identity of the target nucleic acids by sequencing the first bar code and second bar code.

2. A method according to claim 1, wherein the target nucleic acid is either DNA or RNA.

3. A method according to claim 1, wherein both DNA and RNA amplification products are produced from the target nucleic acid sequence.

4. A method according to claim 1, comprising the addition of a reverse transcriptase polymerase and a step of producing cDNA from an RNA target sequence where an RNA target nucleic acid from a single cell is detected and identified.

5. A method according to claim 4, wherein the one or more nucleic acid amplification primer sets provided comprise a DNA specific primer that is blocked before reverse transcriptase is added

6. A method according to claim 5, comprising providing a DNA reverse primer that is blocked during any reverse transcriptase activity so that cDNA is only extended by an RNA reverse primer.

7. A method according to claim 1, comprising a DNA reverse primer that is outside of the RNA reverse primer so that cDNA is only extended by an RNA reverse primer.

8. A method according to claim 1, wherein the target nucleic acid may comprise both DNA and RNA, and either DNA or RNA is selectively amplified to form an amplicon product specific for either a DNA or an RNA target nucleic acid.

9. A method according to claim 1, wherein the protease in step iii) is inactivated by heat after a cell lysate is formed.

10. A method according to claim 1, wherein DNA or RNA amplicons are attenuated, limited, or prevented during amplification by using competimers that selectively modulate DNA or RNA amplicon amplification.

11. A method according to claim 1, wherein DNA or RNA amplicons are attenuated, limited, or prevented during amplification by using biotinylated primers that selectively amplify DNA or RNA amplicons.

12. A method according to claim 1, wherein a portion of library primers provided for RNA amplification comprise uracil and enable the removal RNA amplicons by cleavage.

13. A method according to claim 1, wherein in step iv) each primer set comprises a forward primer and a reverse primer that are complementary to a target nucleic acid or the complement thereof.

14. A method according to claim 12, where a forward primer comprises an identification barcode sequence.

15. A method for detection of a target nucleic acid from a single cell, the method comprising, independent of order presented, the following:
i) selecting one or more target nucleic acid sequence of interest in an individual cell, wherein the target nucleic acid sequence is complementary to a cellular DNA and an RNA in a cell;
ii) providing a sample having a plurality of individual single cells; encapsulating one or more individual cell(s) in a reaction mixture comprising a protease;
iii) incubating the encapsulated cell with the protease in the drop to produce a cell lysate;
iv) providing one or more nucleic acid amplification primer sets complementary to one or more target nucleic acid, wherein at least one primer of a nucleic acid amplification primer set comprises a barcode identification sequence and wherein one or more nucleic acid amplification primer sets provided comprise a DNA specific primer;
v) adding a reverse transcriptase polymerase and producing cDNA from an RNA target; and
vi) performing a nucleic acid amplification reaction to form an amplification product from the nucleic acid of a single cell, said amplification product comprising amplicons of one or more target nucleic acid sequence.

16. A method according to claim 15, further comprising providing an affinity reagent that comprises a nucleic acid sequence complementary to the identification barcode sequence of one of more nucleic acid primer of a primer set, wherein said affinity reagent comprising said nucleic acid sequence complementary to the identification barcode sequence is capable of binding to a nucleic acid amplification primer set comprising a barcode identification sequence.

17. A method according to claim 16, further comprising contacting an affinity reagent to the amplification product comprising amplicons of one or more target nucleic acid sequence under conditions sufficient for binding of the affinity reagent to the target nucleic acid to form an affinity reagent bound target nucleic acid and determining the identity of the target nucleic acids by sequencing the first bar code and second bar code.

18. A method of primer design for selective detection of nucleic acids in a sample comprising both cellular DNA and RNA, the method comprising: i) selecting a target nucleic acid sequence of interest in an individual cell, wherein the target nucleic acid sequence is complementary to a RNA of potential interest that has a corresponding cellular DNA of potential interest; ii) selecting and providing a DNA reverse primer that is blocked to be incapable of priming and extension by reverse transcriptase; iii) selecting and providing one or more nucleic acid amplification primer sets complementary to one or more target nucleic acid, wherein at least one primer of a nucleic acid amplification primer set comprises a barcode identification sequence and wherein one or more nucleic acid amplification primer sets provided comprise a DNA specific primer; iv) optionally, selecting and providing a DNA reverse primer that is outside of the RNA reverse primer in a target nucleic acid region to be amplified; and v) optionally, selecting and providing competing competimer primers that selectively amplify DNA or RNA amplicons.

19. A method according to claim 18, wherein a forward primer comprises an identification barcode sequence.

20. A method according to claim 18, wherein the primers are designed to amplify both DNA and RNA target nucleic acid sequences.


SEQUENCE LISTING (108 sequences)

SEQ ID NO: 1 - DNA, 25 bp, Artificial Sequence
  Description of Artificial Sequence Synthetic primer
caaatgaaaaccaagagaaagaggc

SEQ ID NO: 2 - DNA, 23 bp, Artificial Sequence
  Description of Artificial Sequence Synthetic primer
ggatgtcctcaaagacttggtgt

SEQ ID NO: 3 - DNA, 30 bp, Artificial Sequence
  Description of Artificial Sequence Synthetic primer
gtaaatacattcttcataccaggaccagag

SEQ ID NO: 4 - DNA, 24 bp, Artificial Sequence
  Description of Artificial Sequence Synthetic primer
ggatgtcctcaaaagacttggtgt

SEQ ID NO: 5 - DNA, 14 bp, Artificial Sequence
  Description of Artificial Sequence Synthetic primer
tttgcagggtatta

SEQ ID NO: 6 - DNA, 13 bp, Artificial Sequence
  Description of Artificial Sequence Synthetic primer
cctgttggacatc

SEQ ID NO: 7 - DNA, 13 bp, Artificial Sequence
  Description of Artificial Sequence Synthetic primer
caccatgatgtgc

SEQ ID NO: 8 - DNA, 24 bp, Artificial Sequence
  Description of Artificial Sequence Synthetic primer
gtctttccccacaatcatactgct

SEQ ID NO: 9 - DNA, 20 bp, Artificial Sequence
  Description of Artificial Sequence Synthetic primer
gcgcctttcgagcagtactc

SEQ ID NO: 10 - DNA, 20 bp, Artificial Sequence
  Description of Artificial Sequence Synthetic primer
agacgttggaatgcggggac

SEQ ID NO: 11 - DNA, 22 bp, Artificial Sequence
  Description of Artificial Sequence Synthetic primer
ttctcaatgatgcttggctctg

SEQ ID NO: 12 - DNA, 22 bp, Artificial Sequence
  Description of Artificial Sequence Synthetic primer
ggctgtcccagaatgcaagaag

SEQ ID NO: 13 - DNA, 23 bp, Artificial Sequence
  Description of Artificial Sequence Synthetic primer
atctgttgtaccactccttccct

SEQ ID NO: 14 - DNA, 20 bp, Artificial Sequence
  Description of Artificial Sequence Synthetic primer
agcgaagttccagcagtgtc

SEQ ID NO: 15 - DNA, 21 bp, Artificial Sequence
  Description of Artificial Sequence Synthetic primer
acagagtagaagtggtcagcc

SEQ ID NO: 16 - DNA, 22 bp, Artificial Sequence
  Description of Artificial Sequence Synthetic primer
ggaagcaggtggtcattgatgg

SEQ ID NO: 17 - DNA, 25 bp, Artificial Sequence
  Description of Artificial Sequence Synthetic primer
aaaccatctttcgtttcctttagcc

SEQ ID NO: 18 - DNA, 22 bp, Artificial Sequence
  Description of Artificial Sequence Synthetic primer
gctctgtgtagctgtctctcca

SEQ ID NO: 19 - DNA, 21 bp, Artificial Sequence
  Description of Artificial Sequence Synthetic primer
gcacactattctgccccagga

SEQ ID NO: 20 - DNA, 25 bp, Artificial Sequence
  Description of Artificial Sequence Synthetic primer
catgctgtttaattgtgtggaagat

SEQ ID NO: 21 - DNA, 25 bp, Artificial Sequence
  Description of Artificial Sequence Synthetic primer
caagcaatggatgatttgatgctgt

SEQ ID NO: 22 - DNA, 30 bp, Artificial Sequence
  Description of Artificial Sequence Synthetic primer
aactgaccttaaaatttggagaaaagtatc

SEQ ID NO: 23 - DNA, 25 bp, Artificial Sequence
  Description of Artificial Sequence Synthetic primer
tccagtacattttcattgcccattg

SEQ ID NO: 24 - DNA, 20 bp, Artificial Sequence
  Description of Artificial Sequence Synthetic primer
agctggcaatcgaacgactc

SEQ ID NO: 25 - DNA, 24 bp, Artificial Sequence
  Description of Artificial Sequence Synthetic primer
agctgacctagttccaatcttttc

SEQ ID NO: 26 - DNA, 14 bp, Artificial Sequence
  Description of Artificial Sequence Synthetic primer
caatggatgatctg

SEQ ID NO: 27 - DNA, 13 bp, Artificial Sequence
  Description of Artificial Sequence Synthetic primer
ctgcactgagtct

SEQ ID NO: 28 - DNA, 12 bp, Artificial Sequence
  Description of Artificial Sequence Synthetic primer
atcctcgctggt

SEQ ID NO: 29 - DNA, 15 bp, Artificial Sequence
  Description of Artificial Sequence Synthetic primer
gaacaatggttcact

SEQ ID NO: 30 - DNA, 13 bp, Artificial Sequence
  Description of Artificial Sequence Synthetic primer
gcaaatgctatcg

SEQ ID NO: 31 - DNA, 13 bp, Artificial Sequence
  Description of Artificial Sequence Synthetic primer
catcactggcttt

SEQ ID NO: 32 - DNA, 15 bp, Artificial Sequence
  Description of Artificial Sequence Synthetic primer
ctatcaagtgaactg

SEQ ID NO: 33 - DNA, 49 bp, Artificial Sequence
  Description of Artificial Sequence Synthetic primer
aagcagtggtatcaacgcagagtagggatgtcctcaaaagacttggtgt

SEQ ID NO: 34 - DNA, 49 bp, Artificial Sequence
  Description of Artificial Sequence Synthetic primer
aagcagtggtatcaacgcagagtaggtctttccccacaatcatactgct

SEQ ID NO: 35 - DNA, 45 bp, Artificial Sequence
  Description of Artificial Sequence Synthetic primer
aagcagtggtatcaacgcagagtaggcgcctttcgagcagtactc

SEQ ID NO: 36 - DNA, 45 bp, Artificial Sequence
  Description of Artificial Sequence Synthetic primer
aagcagtggtatcaacgcagagtagagacgttggaatgcggggac

SEQ ID NO: 37 - DNA, 47 bp, Artificial Sequence
  Description of Artificial Sequence Synthetic primer
aagcagtggtatcaacgcagagtagttctcaatgatgcttggctctg

SEQ ID NO: 38 - DNA, 47 bp, Artificial Sequence
  Description of Artificial Sequence Synthetic primer
aagcagtggtatcaacgcagagtagggctgtcccagaatgcaagaag

SEQ ID NO: 39 - DNA, 55 bp, Artificial Sequence
  Description of Artificial Sequence Synthetic primer
aagcagtggtatcaacgcagagtaggtaaatacattcttcataccaggaccagag

SEQ ID NO: 40 - DNA, 48 bp, Artificial Sequence
  Description of Artificial Sequence Synthetic primer
aagcagtggtatcaacgcagagtagatctgttgtaccactccttccct

SEQ ID NO: 41 - DNA, 45 bp, Artificial Sequence
  Description of Artificial Sequence Synthetic primer
aagcagtggtatcaacgcagagtagagcgaagttccagcagtgtc

SEQ ID NO: 42 - DNA, 46 bp, Artificial Sequence
  Description of Artificial Sequence Synthetic primer
aagcagtggtatcaacgcagagtagacagagtagaagtggtcagcc

SEQ ID NO: 43 - DNA, 56 bp, Artificial Sequence
  Description of Artificial Sequence Synthetic primer
gtctcgtgggctcggagatgtgtataagagacagggaagcaggtggtcattgatgg

SEQ ID NO: 44 - DNA, 59 bp, Artificial Sequence
  Description of Artificial Sequence Synthetic primer
gtctcgtgggctcggagatgtgtataagagacagaaaccatctttcgtttcctttagcc

SEQ ID NO: 45 - DNA, 56 bp, Artificial Sequence
  Description of Artificial Sequence Synthetic primer
gtctcgtgggctcggagatgtgtataagagacaggctctgtgtagctgtctctcca

SEQ ID NO: 46 - DNA, 55 bp, Artificial Sequence
  Description of Artificial Sequence Synthetic primer
gtctcgtgggctcggagatgtgtataagagacaggcacactattctgccccagga

SEQ ID NO: 47 - DNA, 59 bp, Artificial Sequence
  Description of Artificial Sequence Synthetic primer
gtctcgtgggctcggagatgtgtataagagacagcatgctgtttaattgtgtggaagat

SEQ ID NO: 48 - DNA, 59 bp, Artificial Sequence
  Description of Artificial Sequence Synthetic primer
gtctcgtgggctcggagatgtgtataagagacagcaagcaatggatgatttgatgctgt

SEQ ID NO: 49 - DNA, 64 bp, Artificial Sequence
  Description of Artificial Sequence Synthetic primer
gtctcgtgggctcggagatgtgtataagagacagaactgaccttaaaatttggagaaaagtatc

SEQ ID NO: 50 - DNA, 59 bp, Artificial Sequence
  Description of Artificial Sequence Synthetic primer
gtctcgtgggctcggagatgtgtataagagacagtccagtacattttcattgcccattg

SEQ ID NO: 51 - DNA, 54 bp, Artificial Sequence
  Description of Artificial Sequence Synthetic primer
gtctcgtgggctcggagatgtgtataagagacagagctggcaatcgaacgactc

SEQ ID NO: 52 - DNA, 58 bp, Artificial Sequence
  Description of Artificial Sequence Synthetic primer
gtctcgtgggctcggagatgtgtataagagacagagctgacctagttccaatcttttc

SEQ ID NO: 53 - DNA, 47 bp, Artificial Sequence
  Description of Artificial Sequence Synthetic primer
gtctcgtgggctcggagatgtgtataagagacagcctgttggacatc

SEQ ID NO: 54 - DNA, 48 bp, Artificial Sequence
  Description of Artificial Sequence Synthetic primer
gtctcgtgggctcggagatgtgtataagagacagcaatggatgatctg

SEQ ID NO: 55 - DNA, 47 bp, Artificial Sequence
  Description of Artificial Sequence Synthetic primer
gtctcgtgggctcggagatgtgtataagagacagctgcactgagtct

SEQ ID NO: 56 - DNA, 46 bp, Artificial Sequence
  Description of Artificial Sequence Synthetic primer
gtctcgtgggctcggagatgtgtataagagacagatcctcgctggt

SEQ ID NO: 57 - DNA, 47 bp, Artificial Sequence
  Description of Artificial Sequence Synthetic primer
gtctcgtgggctcggagatgtgtataagagacagcaccatgatgtgc

SEQ ID NO: 58 - DNA, 49 bp, Artificial Sequence
  Description of Artificial Sequence Synthetic primer
gtctcgtgggctcggagatgtgtataagagacaggaacaatggttcact

SEQ ID NO: 59 - DNA, 47 bp, Artificial Sequence
  Description of Artificial Sequence Synthetic primer
gtctcgtgggctcggagatgtgtataagagacaggcaaatgctatcg

SEQ ID NO: 60 - DNA, 47 bp, Artificial Sequence
  Description of Artificial Sequence Synthetic primer
gtctcgtgggctcggagatgtgtataagagacagcatcactggcttt

SEQ ID NO: 61 - DNA, 49 bp, Artificial Sequence
  Description of Artificial Sequence Synthetic primer
gtctcgtgggctcggagatgtgtataagagacagctatcaagtgaactg

SEQ ID NO: 62 - DNA, 48 bp, Artificial Sequence
  Description of Artificial Sequence Synthetic primer
gtctcgtgggctcggagatgtgtataagagacagtttgcagggtatta

SEQ ID NO: 63 - DNA, 45 bp, Artificial Sequence
  Description of Artificial Sequence Synthetic primer
gtctcgtgggctcggagatgtgtataagagacagtgttggacatc

SEQ ID NO: 64 - DNA, 46 bp, Artificial Sequence
  Description of Artificial Sequence Synthetic primer
gtctcgtgggctcggagatgtgtataagagacagatggatgatctg

SEQ ID NO: 65 - DNA, 45 bp, Artificial Sequence
  Description of Artificial Sequence Synthetic primer
gtctcgtgggctcggagatgtgtataagagacaggcactgagtct

SEQ ID NO: 66 - DNA, 44 bp, Artificial Sequence
  Description of Artificial Sequence Synthetic primer
gtctcgtgggctcggagatgtgtataagagacagcctcgctggt

SEQ ID NO: 67 - DNA, 45 bp, Artificial Sequence
  Description of Artificial Sequence Synthetic primer
gtctcgtgggctcggagatgtgtataagagacagccatgatgtgc

SEQ ID NO: 68 - DNA, 47 bp, Artificial Sequence
  Description of Artificial Sequence Synthetic primer
gtctcgtgggctcggagatgtgtataagagacagacaatggttcact

SEQ ID NO: 69 - DNA, 45 bp, Artificial Sequence
  Description of Artificial Sequence Synthetic primer
gtctcgtgggctcggagatgtgtataagagacagaaatgctatcg

SEQ ID NO: 70 - DNA, 45 bp, Artificial Sequence
  Description of Artificial Sequence Synthetic primer
gtctcgtgggctcggagatgtgtataagagacagtcactggcttt

SEQ ID NO: 71 - DNA, 47 bp, Artificial Sequence
  Description of Artificial Sequence Synthetic primer
gtctcgtgggctcggagatgtgtataagagacagatcaagtgaactg

SEQ ID NO: 72 - DNA, 46 bp, Artificial Sequence
  Description of Artificial Sequence Synthetic primer
gtctcgtgggctcggagatgtgtataagagacagtgcagggtatta

SEQ ID NO: 73 - DNA, 22 bp, Artificial Sequence
  Description of Artificial Sequence Synthetic primer
ccatcaatgaccacctgcttcc

SEQ ID NO: 74 - DNA, 25 bp, Artificial Sequence
  Description of Artificial Sequence Synthetic primer
ggctaaaggaaacgaaagatggttt

SEQ ID NO: 75 - DNA, 22 bp, Artificial Sequence
  Description of Artificial Sequence Synthetic primer
tggagagacagctacacagagc

SEQ ID NO: 76 - DNA, 21 bp, Artificial Sequence
  Description of Artificial Sequence Synthetic primer
tcctggggcagaatagtgtgc

SEQ ID NO: 77 - DNA, 25 bp, Artificial Sequence
  Description of Artificial Sequence Synthetic primer
atcttccacacaattaaacagcatg

SEQ ID NO: 78 - DNA, 25 bp, Artificial Sequence
  Description of Artificial Sequence Synthetic primer
acagcatcaaatcatccattgcttg

SEQ ID NO: 79 - DNA, 30 bp, Artificial Sequence
  Description of Artificial Sequence Synthetic primer
gatacttttctccaaattttaaggtcagtt

SEQ ID NO: 80 - DNA, 25 bp, Artificial Sequence
  Description of Artificial Sequence Synthetic primer
caatgggcaatgaaaatgtactgga

SEQ ID NO: 81 - DNA, 20 bp, Artificial Sequence
  Description of Artificial Sequence Synthetic primer
gagtcgttcgattgccagct

SEQ ID NO: 82 - DNA, 24 bp, Artificial Sequence
  Description of Artificial Sequence Synthetic primer
gaaaagattggaactaggtcagct

SEQ ID NO: 83 - DNA, 50 bp, Artificial Sequence
  Description of Artificial Sequence Synthetic primer
aagcagtggtatcaacgcagagtagggatgtcctcaaaagacttggtgtc

SEQ ID NO: 84 - DNA, 50 bp, Artificial Sequence
  Description of Artificial Sequence Synthetic primer
aagcagtggtatcaacgcagagtaggtctttccccacaatcatactgctc

SEQ ID NO: 85 - DNA, 47 bp, Artificial Sequence
  Description of Artificial Sequence Synthetic primer
aagcagtggtatcaacgcagagtaggcgcctttcgagcagtactcac

SEQ ID NO: 86 - DNA, 46 bp, Artificial Sequence
  Description of Artificial Sequence Synthetic primer
aagcagtggtatcaacgcagagtagagacgttggaatgcggggacc

SEQ ID NO: 87 - DNA, 48 bp, Artificial Sequence
  Description of Artificial Sequence Synthetic primer
aagcagtggtatcaacgcagagtagttctcaatgatgcttggctctgc

SEQ ID NO: 88 - DNA, 49 bp, Artificial Sequence
  Description of Artificial Sequence Synthetic primer
aagcagtggtatcaacgcagagtagggctgtcccagaatgcaagaagac

SEQ ID NO: 89 - DNA, 56 bp, Artificial Sequence
  Description of Artificial Sequence Synthetic primer
aagcagtggtatcaacgcagagtaggtaaatacattcttcataccaggaccagagc

SEQ ID NO: 90 - DNA, 49 bp, Artificial Sequence
  Description of Artificial Sequence Synthetic primer
aagcagtggtatcaacgcagagtagatctgttgtaccactccttccctc

SEQ ID NO: 91 - DNA, 46 bp, Artificial Sequence
  Description of Artificial Sequence Synthetic primer
aagcagtggtatcaacgcagagtagagcgaagttccagcagtgtcc

SEQ ID NO: 92 - DNA, 47 bp, Artificial Sequence
  Description of Artificial Sequence Synthetic primer
aagcagtggtatcaacgcagagtagacagagtagaagtggtcagccc

SEQ ID NO: 93 - DNA, 57 bp, Artificial Sequence
  Description of Artificial Sequence Synthetic primer
gtctcgtgggctcggagatgtgtataagagacagggaagcaggtggtcattgatggc

SEQ ID NO: 94 - DNA, 60 bp, Artificial Sequence
  Description of Artificial Sequence Synthetic primer
gtctcgtgggctcggagatgtgtataagagacagaaaccatctttcgtttcctttagccc

SEQ ID NO: 95 - DNA, 57 bp, Artificial Sequence
  Description of Artificial Sequence Synthetic primer
gtctcgtgggctcggagatgtgtataagagacaggctctgtgtagctgtctctccac

SEQ ID NO: 96 - DNA, 56 bp, Artificial Sequence
  Description of Artificial Sequence Synthetic primer
gtctcgtgggctcggagatgtgtataagagacaggcacactattctgccccaggac

SEQ ID NO: 97 - DNA, 61 bp, Artificial Sequence
  Description of Artificial Sequence Synthetic primer
gtctcgtgggctcggagatgtgtataagagacagcatgctgtttaattgtgtggaagatac

SEQ ID NO: 98 - DNA, 61 bp, Artificial Sequence
  Description of Artificial Sequence Synthetic primer
gtctcgtgggctcggagatgtgtataagagacagcaagcaatggatgatttgatgctgtac

SEQ ID NO: 99 - DNA, 65 bp, Artificial Sequence
  Description of Artificial Sequence Synthetic primer
gtctcgtgggctcggagatgtgtataagagacagaactgaccttaaaatttggagaaaagtatcc

SEQ ID NO: 100 - DNA, 60 bp, Artificial Sequence
  Description of Artificial Sequence Synthetic primer
gtctcgtgggctcggagatgtgtataagagacagtccagtacattttcattgcccattgc

SEQ ID NO: 101 - DNA, 55 bp, Artificial Sequence
  Description of Artificial Sequence Synthetic primer
gtctcgtgggctcggagatgtgtataagagacagagctggcaatcgaacgactcc

SEQ ID NO: 102 - DNA, 59 bp, Artificial Sequence
  Description of Artificial Sequence Synthetic primer
gtctcgtgggctcggagatgtgtataagagacagagctgacctagttccaatcttttcc

SEQ ID NO: 103 - DNA, 170 bp, Homo sapiens
  modified_base (1)..(150): a, c, t, g, unknown or other
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngtccccgcattccaacgtct

SEQ ID NO: 104 - DNA, 164 bp, Homo sapiens
  modified_base (1)..(150): a, c, t, g, unknown or other
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaccagcgaggatgg

SEQ ID NO: 105 - DNA, 14 bp, Artificial Sequence
  Description of Artificial Sequence Synthetic primer
ccatcctcgctggt

SEQ ID NO: 106 - DNA, 132 bp, Homo sapiens
  modified_base (1)..(110): a, c, t, g, unknown or other
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncagagccaagcatcattgagaa

SEQ ID NO: 107 - DNA, 124 bp, Homo sapiens
  modified_base (1)..(110): a, c, t, g, unknown or other
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngcacatcatggtgg

SEQ ID NO: 108 - DNA, 14 bp, Artificial Sequence
  Description of Artificial Sequence Synthetic primer
ccaccatgatgtgc